(19) United States
(12) Patent Application Publication
(10) Pub. No.: US 2016/0346202 A1
(43) Pub. Date: Dec. 1, 2016

(54) Composition for Vectorizing an Anti-Cancer Agent

(71) Applicant: GUERBET, Villepinte (FR)

(72) Inventors: Robic Caroline, Nogent Sur Marne (FR); Mayer Jean-François, Aulnay Sous Bois (FR)

(21) Appl. No.: 15/117,109

(22) PCT Filed: Feb. 6, 2015
(86) PCT No.: PCT/EP2015/052527

§ 371 Date: Aug. 5, 2016

(30) Foreign Application Priority Data
Feb. 7, 2014 (FR) 14 50972

(51) Int. Cl. A61K 9/107; A61K 31/704; A61K 33/26; A61K 31/407; A61K 51/12; A61K 47/44; A61K 47/14

(52) U.S. Cl. CPC A61K 9/107; A61K 47/44; A61K 31/704; A61K 47/14; A61K 31/407; A61K 51/1217; A61K 33/26

(57) ABSTRACT

The invention relates to a stable water-in-oil emulsion composition containing: 20% to 40% (v/v) aqueous phase in the form of droplets and containing an anti-cancer agent; and 60% to 80% (v/v) lipid phase containing an iodized oil and at least one surfactant, having Formula (I) as follows, in a proportion, of surfactant mass relative to the total volume of the composition, of 0.3% to 5%:

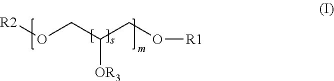

wherein s is 0 or 1, m is a whole number from 2 to 30, R1 has Formula (II) as follows, and R2 and R3 are independently H or R1:

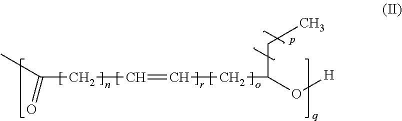

wherein n is a whole number from 4 to 10, o is a whole number from 1 to 4, p is a whole number from 3 to 7, q is a whole number from 2 to 10, and r has a value of 0 or 1.

The present invention relates to a composition for vectorizing an anti-cancer agent, comprising an iodized oil and a surfactant, that is of use for preparing a composition in the form of a water-in-oil emulsion comprising an anti-cancer agent, an iodized oil and this surfactant.

For more than a century, iodized oils such as the product Lipiodol® have been used as contrast products in radiological examinations such as lymphography or for the diagnosis of hepatic lesions. Lipiodol® consists mainly of ethyl esters of iodized fatty acids of poppy oil.

For more than thirty years, these iodized oils have been used in interventional radiology procedures. Lipiodol® is characterized by its propensity to be selectively taken up by hepatic tumors. It has therefore been proposed as an anti-cancer agent vector for the treatment of hepatocellular carcinoma in a technique which is called TransArterial ChemoEmbolization (TACE) (Nakamura et al.: Radiology, 1989; 170:783-6 and J. M. Idée-B. Guiu: Critical Reviews in Oncology/Hematology, 2013; 88(3):530-49). Iodized oils, and in particular Lipiodol®, are also known to induce transient embolization of the arterial circulation, thus causing a slowing down thereof. Given that most anti-cancer agents are water-soluble, the “emulsion” form, which is suitable for mixing two phases not soluble in one another, appears to be the most judicious for mixing an iodized oil and an anti-cancer agent. It appears to be the most suitable for transporting and delivering, to a tumor, an anti-cancer agent which is too toxic and not effective enough when it is administered non-emulsified intra-arterially or systemically.

A “water-in-oil” emulsion, termed “inverse” emulsion, is an emulsion which is denoted W/O (water-in-oil). It is a dispersion of droplets of aqueous phase in a lipid phase. An “oil-in-water” emulsion is a “direct” emulsion that is denoted O/W (oil-in-water). Unlike W/O emulsions, it is then a dispersion of droplets of lipid phase in an aqueous phase. The term “sense of the emulsion” is used when referring to the W/O or O/W nature of an emulsion.

Oil-in-water (O/WV) emulsions, which comprise the anti-cancer agent in the aqueous continuous phase, have the considerable drawback of rapidly releasing the anti-cancer agent in the blood. A not insignificant part of the therapeutic agent does not therefore reach the targeted site, which may, on the one hand, induce systematic toxicity and, on the other hand, reduce the efficacy of this therapeutic agent. Furthermore, this type of O/W emulsion has the risk of causing a pulmonary or even cerebral embolism. This risk is increased when the size of the droplets of oil of these emulsions is less than 10 μm. This second drawback is difficult to exclude since, when increasing the size of the droplets, the instability of these emulsions is increased.

Water-in-oil (W/O) emulsions, also called “inverse emulsions”, and comprising an iodized oil and an anti-cancer agent, are less commonly mentioned in the literature than O/W emulsions. They are described as releasing the therapeutic more slowly in the tumor and as having a higher viscosity than oil-in-water emulsions (De Baere et al., Radiology 1995; 194:165-170). These reasons lead to the choice of a form of W/O emulsion for vectorizing an anti-cancer agent within a tumor. However, these W/O emulsions are not always sufficiently effective because of their lack of stability on contact with the blood and the vascular bifurcations upstream of the tumor. Indeed, in order to increase the tumor targeting of anti-cancer agents and to at the same time improve the therapeutic efficacy and the tolerance of the treatment, an emulsion must remain stable up to the moment it reaches the tumor, and its distribution in the tumor lesion must be complete and uniform.

Various solutions for stabilizing emulsions have thus been proposed in the prior art. Numerous authors have proposed the use of surfactant with a high HLB (more than 8) for stabilizing O/W emulsions.

The use of surfactant with a high or even very high HLB, such as the polyoxyethylenated fatty acid esters of sorbitan, polyoxyethylenated sorbitan monostearate or polysorbate 60 (Montanox® 60, HLB=14.9) and polyoxyethylenated sorbitan monolaurate or polysorbate 20 (Montanox 20®, HLB=16.7), has been described for preparing oil-in-water emulsions based on idarubicin and Lipiodol®, which are stable for 6 months.

JPH0647559 describes an O/W emulsion comprising between 10% and 30% of Lipiodol®, an anti-cancer agent and between 0.1% and 2% of a hydrophilic surfactant, HCO-60, otherwise known as polyoxyethylene hydrogenated castor oil (HLB=14). It is, a priori, a PEG-60 bonded to a ricinoleic acid.

EP 0 294 534 describes a contrast product in emulsion form made from an iodized oil emulsified using organic compounds such as amino acids (phenylalanine, alanine, leucine, isoleucine, glycine, serine or taurine), fatty acids such as pelargonic acid, oleic acid (HLB=17) or linoleic acid (HLB=16) or a liposoluble vitamin such as vitamin E.

EP 0 581 842 describes an oil-in-water emulsion comprising esters of fatty acids which are iodized and derive from poppyseed oil emulsified using a mixture of phospholipids and of cyclopentaphenanthrene derivatives such as sterols.

Applications EP 0 294 534 and EP 0 581 842 refer to other documents. It is in particular discovered that DE 26 02 907 describes an oil-in-water emulsion containing between 50% and 60% of iodized triglycerides, between 2% and 10% of fatty acid esters of polyoxyethylene sorbitan (HLB=13 to 17) and between 2% and 40% of water. Grimes et al. (J. Pharm. Sci. 1979 January; 68(1):52-6) describes the use of polysorbate 80 (HLB=15), of sorbitan monooleate (HLB=8.6) and of phosphatidylcholine for obtaining emulsions comprising iodized oil. Vermess et al. has described emulsions (U.S. Pat. No. 4,404,182 or J. Comput. Assist. Tomogr. 3: 25-31, 1979) containing 53% (v/v) of Lipiodol®, 10% of alcohol and 0.45% of soya lecithin. These oil-in-water emulsions have particles sizes of 2 to 3 μm. Schumacher et al. (Europ. J. Radiol. 5, 167-174, 1985) describes various emulsions containing iodized oils prepared using emulsifiers such as polyoxyethylene-4-sorbitan monolaurate (Tween® 80, Serva: HLB=15.3), glycerol polyethylene glycol ricinoleate (Cremophor® EL: HLB=14.5), diacetylphosphate DP (Sigma), lecithin from eggs (Fluka GmbH), doxypolygelatin (Gelinfundol® 5.5% Biotest GmbH) and dextran 60 (Macrodex™4.5%, R L Knoll). GB 676 738 describes emulsions containing iodized oils and synthetic nonionic emulsifiers such as fatty acid monoesters of polyhydroxy alcohols (monoesters of sorbitol and of lauric acid, of palmitic acid, of stearic acid or of oleic acid, monoesters of glycerol and of fatty acids such as glyceryl monostearate and glyceryl monooleate, monoesters of glycol such as ethylene glycol, tetraethylene glycol or dodecaethylene glycol with fatty acids such as palmitic acid, stearic acid or lauric acid), it being possible for these esters to react with polyalkylene oxides to form polyoxyalkylene derivatives. U.S. Pat. No. 3,356,575 describes an emulsion containing an iodized oil, glycerol and lecithin. U.S. Pat. No. 4,917,880 describes an emulsion comprising 10% of iodized oil and, in the aqueous phase, 1.2% of purified egg phospholipids with 2.25% of glycerol and 0.1% of phenylalanine.

The use of amiodarone (an antiarythmic medicament of chemical formula (2-butyl-3-benzofuranyl)[4-[2-(diethylamino)ethoxy]-3,5-diiodophenyl]methanone) has made it possible to stabilize an oil-in-water emulsion of Lipiodol® (44% (v/v)) and doxorubicin or pirarubicin, for up to four weeks at 37° C. This property is due to the presence, in this medicament, of an excipient, polysorbate 80, which is an emulsifier with a high HLB (Boutin et al., Digestive and Liver Disease 43 (2011) 905-911). Additional studies by the same team have made it possible to show that amiodarone provides virtually no improvement in the stability of an emulsion based on Lipiodol® and idarubicin, and does not appear to increase the cytotoxicity in the anti-cancer agent. The use alone of idarubicin and of Lipiodol® is therefore even recommended.

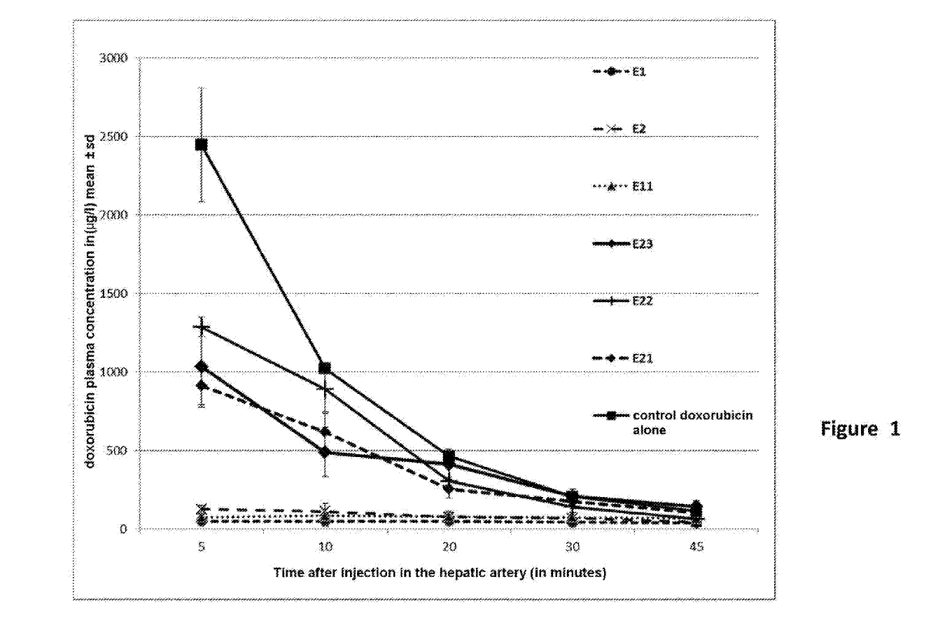

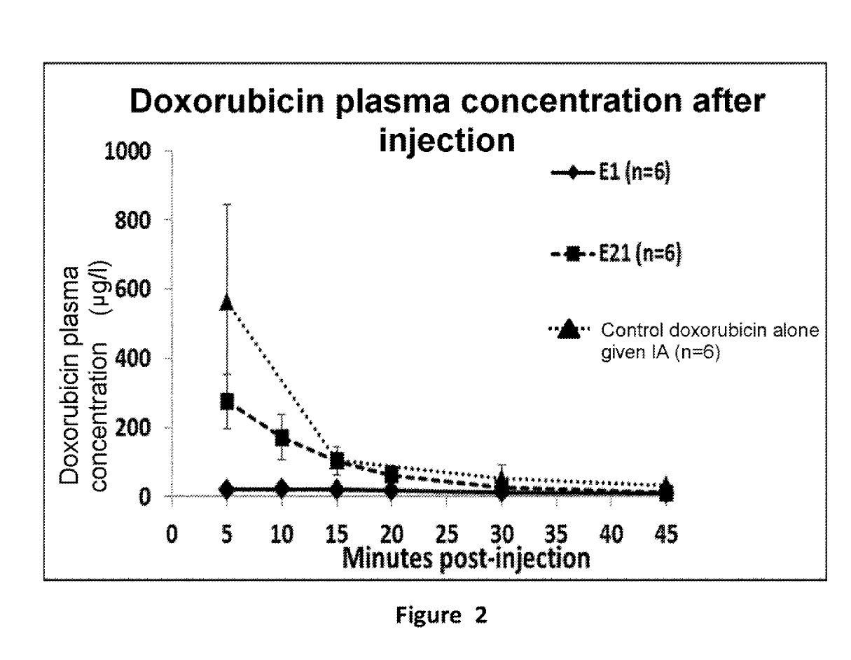

Nakamura et al. (Radiology, 1989; 170:783-6) shows the visual appearance of various emulsions obtained by mixing 1 ml of distilled water comprising an ionic contrast product, meglumine sodium diatrizoate (Hypaque®, Gastrografin® or Urografin®) and 3 ml of Lipiodol® (FIG. 1). It is indicated that the emulsion C did not undergo phase separation after 24 h, but it can be easily noted in this figure that this emulsion is in reality not stable, the lower part of the tube that contains it being clearer than the upper part thereof. This document also describes the preparation of an emulsion of Lipiodol® and of doxorubicin or mitomycin in ratios of 2-3/1. It is indicated that the emulsion obtained is a W/O emulsion. The lowest amount of release of anti-cancer agent in the case of the use of this emulsion is emphasized (FIG. 2). The plasma peak visualized after injection of this emulsions remains, however, not insignificant. This emulsion must not be sufficiently stable since there is no use of surfactant. Indeed, 2 minutes after intra-arterial injection of their emulsion, a plasma concentration of doxorubicin is observed which is 83% lower (((3500−600)/3500)×100) compared with the plasma concentration measured after injection of this anti-cancer agent alone. At 5 minutes, this decrease is 80%.

Raoul et al. (Cancer, 1992, vol. 70, No. 3, 585-90), describes emulsions comprising 50 mg of doxorubicin, made by mixing 10 ml of Lipiodol® and 2.5 ml of ioxaglate (Hexabrix®). The emulsions obtained, the W/O or O/W sense of which is not specified, cause a plasma peak that is significantly lower than that caused by the intra-arterial injection of doxorubicin alone. However, this plasma peak indicates a not insignificant passing of the anti-cancer agent into the blood. Indeed, 2 minutes after intra-arterial injection of these emulsions, a plasma concentration of doxorubicin is observed that is 59% lower (((2200−900)/2200)×100) compared with the plasma concentration measured after injection of this anti-cancer agent alone. The calculation corresponding to 5 minutes after injection is even more unfavorable since this decrease is then only 33% (((1050−700)/1050)×100). When an embolization is performed after injection of these emulsions, these decreases are, at 2 and 5 minutes, respectively 82% (((2200−400)/2200)×100) and 43% (((700−400)/700)×100).

These various emulsions, when they are in “oil-in-water” form, have an insufficient anti-cancer agent-vectorizing capacity, even if they have been stabilized using a surfactant with a high HLB, and their use still exhibits a significant risk of embolism. This insufficient vectorization capacity is explained by the very nature of the emulsion, since, in the case of oil-in-water emulsions, the anti-cancer agent, which is usually water-soluble, is in the aqueous continuous phase and is therefore very rapidly diluted in the blood stream. In addition, several of these emulsions contain synthetic emulsifiers such as Tweens® (high HLB) or Spans® (either lower or higher HLB), which are emulsifiers listed in the European pharmacopoeia, and which cause side effects. Polysorbates such as Tweens® are described as potentially toxic. Sorbitan esters such as Spans® are not recommended for use by parenteral injection (Handbook of Pharmaceutical Excipients, 2009).

Frequently, emulsions described in publications as “water-in-oil” emulsions are not emulsions of this nature. When they are actually in the W/O form, these emulsions have insufficient stabilities and insufficient anti-cancer agent-vectorizing capacities. They therefore have an insufficient efficacy after injection since a considerable part of the amount of anti-cancer agent injected intra-arterially does not reach the targeted lesion (Raoul et al., 1992).

The applicant has developed a composition which makes it possible to prepare a water-in-oil emulsion comprising an anti-cancer agent which is stable for at least 24 h at 20° C. and which generally has an improved vectorizing capacity compared with the prior art emulsions.

This emulsion therefore has two major advantages: it can be easily used in a hospital context, since its stability allows it to be prepared at least 24 hours in advance in the hospital pharmacy, and it presents a very limited risk to the patient, while at the same time having an improved therapeutic efficacy.

This emulsion also has the advantage of making it possible to correlate an amount of iodized oil (e.g. Lipiodol®) present in a tumor, which may be estimated by means of an imaging method, with an amount of anti-cancer agent actually present in the tumor. For the majority of prior art emulsions, the amount of iodized oil that is estimated to be present in a tumor is in no way an indication of the amount of anti-cancer agent present in this tumor. The emulsion according to the invention therefore makes it possible to reduce the false positives when seeking to verify that the anti-cancer agent has effectively been administered at the heart of the tumor.

Thus, a subject of the invention is a composition in the form of a water-in-oil emulsion comprising:
from 20% to 40% (v/v) of aqueous phase, preferentially from 20% to 35% (v/v), more preferentially 25% (v/v), of aqueous phase, in the form of droplets, comprising an anti-cancer agent,
from 60% to 80%, preferentially from 65% to 80% (v/v), more preferentially 75% (v/v), of lipid phase comprising an iodized oil and at least one surfactant of formula (I) in a proportion, by weight of surfactant relative to the total volume of the composition, of 0.3% to 5%, preferentially of 0.5% to 2%, more preferentially of 1%, formula (I) of said surfactant being the following:

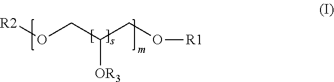

in which:
s is 0 or 1,
m represents an integer from 2 to 30,
R₁ represents a group of formula (II)

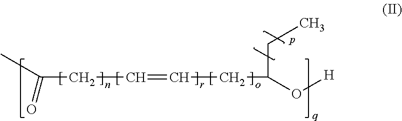

in which n represents an integer from 4 to 10, o represents an integer from 1 to 4, p represents an integer from 3 to 7, q represents an integer from 2 to 10 and r is 0 or 1,
R₂ represents a hydrogen atom or is identical to R₁, and
each R₃ independently represents a hydrogen atom or is identical to R₁.

Preferably, in formula (I) above, each R₃ represents a hydrogen atom. Formula (I) of said surfactant then has the following formula (I′):

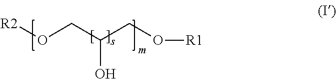

The proportion of surfactant is expressed by weight of surfactant relative to the total volume of the composition in emulsion form. The proportions of aqueous or lipid phases are expressed by volume of the phase relative to the total volume of the composition in emulsion form.

This composition is for vectorizing an anti-cancer agent. The invention also relates to the use of this composition as an anti-cancer agent vector.

This composition is in the form of a water-in-oil emulsion (also known as “inverse emulsion” or W/O emulsion). Such an emulsion consists of a lipid phase and an aqueous phase dispersed in the form of droplets. The iodized oil of the composition is in the lipid phase. The surfactant of formula (I) or (I′) is at the interface between the aqueous and lipid phases. For the purposes of the present application, for the calculation of the proportions of aqueous and lipid phases, it will be considered that the surfactant is in the lipid phase.

In particular, the following embodiments are advantageous:

| | % (v/v) | | |
|---|---|---|---|
| | of aqueous phase | | |
| | in the form of | % (v/v) | |
| | droplets comprising | of lipid phase | % (w/v) |
| | an anti-cancer | comprising an | of at least one |
| | agent | iodized oil | surfactant |
| Composition in | 20-35 | 65-80 | 0.5-2 |
| emulsion form | | | |
| according to the | | | |
| invention | | | |
| Composition in | 25 | 75 | 1 |
| emulsion form | | | |
| according to the | | | |
| invention | | | |

The emulsion according to the invention is advantageously stable. The term “stable emulsion” is intended to mean an emulsion having, under conventional temperature (20° C.) and atmospheric pressure (1 bar) conditions and within 24 hours following its preparation, a visual phase separation of less than 5% by volume relative to the total composition in emulsion form. Preferentially, a “stable emulsion” is intended to mean an emulsion exhibiting no visual phase separation under the conditions mentioned above and within 24 hours following its preparation. Visual phase separation manifests itself when a solution no longer appears uniform to the eye, i.e. when the appearance of at least two phases is observed.

More preferentially, the term “stable emulsion” is intended to mean an emulsion of which the average droplet size varies by less than 10%, in particular by less than 5%, preferably of which the average droplet size does not vary, wherein the average size is measured with an optical microscope (for example the Leica DM2000 LED microscope) 24 hours after its preparation.

Preferably, the intra-arterial injection of the emulsion according to the invention induces a decrease in the plasma concentration of the anti-cancer agent between 0 and 5 minutes following this injection of more than 90%, preferentially of more than 94%, more preferentially of more than 97%, even more preferentially of more than 99%, relative to the intra-arterial injection of the anti-cancer agent alone. Advantageously, these plasma concentrations and this decrease are confirmed by plasma kinetics measurements according to protocols known to those skilled in the art.

The expression of the difference between a plasma concentration peak of an anti-cancer agent after injection of a particular product comprising this agent and that obtained after injection of the anti-cancer agent alone is in particular mentioned by Hong et al. (Clin. Cancer Res. 2006: 12(8)).

When the emulsion comprises less than 20% (v/v) of aqueous phase, the anti-cancer agent is difficult to dissolve therein. When the emulsion comprises more than 40% of aqueous phase, the viscosity of the composition in emulsion form is too high. This is because, when increasing the concentration of droplets of aqueous phase in the lipid continuous phase comprising an iodized oil, the viscosity of the overall composition is increased.

The aqueous phase comprises an anti-cancer agent at a therapeutically effective dose. The term “therapeutically effective dose” is intended to mean a dose which makes it possible to treat a cancer or to slow down the progression thereof. Preferentially, when the anti-cancer agent is chosen from anthracyclines, a therapeutically effective dose represents an amount of anti-cancer agent of from 20 to 150 mg, more preferentially from 50 to 100 mg.

The density of the lipid phase is preferentially from 1.10 to 1.30, more preferentially from 1.20 to 1.30, even more preferentially 1.28. Preferentially, the aqueous phase and the lipid phase have the same density (in other words, they are of equal density) or densities up to 5% different than one another.

In order to increase the density of the aqueous phase, a densifying agent can be added thereto (a densification of this phase comprising an anti-cancer agent is then carried out).

Conversely, in order to decrease the density of the lipid phase comprising an iodized oil, a second oil having a density of less than 1 can be added (a “dedensification” of the lipid phase comprising an iodized oil is then carried out).

In one advantageous embodiment, the aqueous phase can thus also comprise a densifying agent, preferentially at least one nonionic iodinated contrast product. The nonionic iodinated product, that can be used as a densifying agent, is preferably chosen from iobitridol (Xenetix®), iopamidol (Iopamiron®, Isovue®), iomeprol (Iomeron®), ioversol (Optiray®, Optiject®), iohexol (Omnipaque®), iopentol (Imagopaque®), ioxitol (Oxilan®), iopromide (Ultravist®), metrizamide (Amipaque®), iosarcol (Melitrast®), iotrolan (Isovist®), iodixanol (Visipaque®), iosimenol and iosimide (Univist®) and a mixture thereof. Iobitridol is the preferential nonionic iodinated product. The Xenetix® 250 and Xenetix® 300 products have densities of 1.28 and 1.34, respectively. These nonionic iodinated contrast products have the advantage of allowing good solubility of the anti-cancer agent in the aqueous phase and of not destabilizing the emulsion.

The use of ionic iodinated contrast products such as ioxaglic acid (Hexabrix®) or meglumina and/or sodium diatrizoate (Hypaque®, Gastrografin®, Gastroview® or Urografin®) is not indicated since these contrast products have the drawback of reducing the solubility of the anti-cancer agent in the aqueous phase, or even of preventing the dissolution thereof and/or of increasing the osmolality of the compositions.

In another advantageous embodiment (which may or may not be combined with the embodiment above in which the aqueous phase comprises a densifying agent), the lipid phase may also comprise at least one non-iodized oil having a density of less than 1, preferably a non-iodized oil having a density of less than 0.96, even more preferentially a non-iodized oil chosen from linseed oil, soybean oil, palm oil, coconut oil, caster oil, corn oil, cottonseed oil, peanut oil, sesame oil, sunflower oil, safflower oil, almond oil, olive oil, poppy oil and an oil comprising or consisting of a mixture of fatty acid triglycerides of formula:

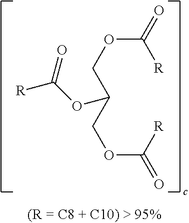

wherein R is an aliphatic chain comprising from 3 to 35 carbon atoms, with the proviso that more than 95% of said fatty acids are C8 and/or 010, sold for example under the name Miglyol®, for example the oil Miglyol® 810, the oil Miglyol® 812 (caprylic/capric triglyceride), the oil Miglyol® 818 (caprylic/capric/linoleic triglyceride), the oil Miglyol® 612 (glyceryl trihexanoate) or other propylene glycol dicaprylate dicaprate Miglyol® derivatives. The expression (R=08+010)>95% signifies that the triglycerides of the mixture are triglycerides of fatty acids of which more than 95% are C8 and/or 010 fatty acids (capric or caprylic acid). When the fatty acid is a C8 fatty acid, R is a chain comprising 7 carbon atoms and when the fatty acid is a 010 fatty acid, R is a chain comprising 9 carbon atoms.

The densities of the various non-iodized oils listed are specified in the following table:

| Oil name | Density |
|---|---|
| Linseed oil | 0.94 |
| Soybean oil | 0.92 |
| Miglyol ® oil | 0.94 |
| Palm oil | 0.90 |
| Coconut oil | 0.92 |
| Caster oil | 0.96 |
| Corn oil | 0.90 |
| Cottonseed oil | 0.92 |
| Peanut oil | 0.92 |
| Sesame oil | 0.92 |
| Sunflower oil | 0.93 |
| Safflower oil | 0.92 |
| Almond oil | 0.91 |
| Olive oil | 0.915 |
| Poppy oil | 0.928 |

In this precise embodiment, the density of the lipid phase comprising the iodized oil and one or more non-iodized oils as defined above is then preferentially from 0.9 to 1.2, more preferentially from 0.95 to 1.10, even more preferentially 1.05.

The size of the aqueous phase droplets is preferentially included from 1 to 200 μm, more preferentially included from 5 to 100 μm, even more preferentially included from 5 to 50 μm, or even from 5 to 10 μm. This size even further improves the stability of the emulsion. The size can be measured using an optical microscope (for example, the Leica DM2000 LED microscope).

Preferentially, the aqueous phase droplets are uniformly distributed. The uniformity is verified using an optical microscope: if aggregates of droplets are observed, these droplets are not uniformly distributed.

The aqueous phase/lipid phase volume ratio in the composition in the form of an emulsion according to the invention is advantageously from ½ (i.e. 0.5) to ¼ (i.e. 0.25), preferentially from ⅖ (i.e. 0.4) to 3/10 (i.e. 0.3), more preferentially ⅓ (i.e. approximately 0.33). A ratio of less than ½ makes it possible to definitely obtain a W/O emulsion. Indeed, a 1/1 ratio between the lipid phase and the aqueous phase naturally promotes an O/W sense. In order to force the W/O sense, the amount of iodized oil added must be increased. Above a ¼ ratio, the risk of embolism becomes significant. This is because, in order to dissolve a therapeutically effective amount of anti-cancer agent in the aqueous phase, it is necessary for this aqueous phase to have a sufficient volume. Having a lipid phase comprising an iodized oil and which is more than 4 times greater in volume than the aqueous phase generally results in the dose of iodized oil used becoming greater than the authorized limit. In the legal notices regarding a product such as Lipiodol®, it is indicated that the volume injected in an interventional radiology procedure must not exceed 15 ml.

The volume percentages of aqueous and lipid phases and the aqueous phase/lipid phase volume ratio of the composition in the form of an emulsion according to the invention make it possible to systematically obtain an inverse (W/O) emulsion which makes it possible to improve the conveying of an anti-cancer agent into a tumor.

Advantageously, the composition according to the invention has a viscosity at 20° C. included from 100 to 200 mPa·s, preferentially included from 120 to 170 mPa·s, more preferentially included from 150 to 165 mPa·s, and/or a viscosity at 37° C. included from 40 to 80 mPa·s, preferentially included from 50 to 70 mPa·s, more preferentially included from 60 to 70 mPa·s. The viscosity values are obtained using a Malvern Instruments Kinexus Pro rheometer, having a 4° cone-plate cell with a diameter of 40 mm. The measurements are carried out at an imposed stress in a range of from 0.16 to 10 Pa.

### Iodized Oils

The term “fatty acid” is intended to denote saturated or unsaturated, aliphatic carboxylic acids having a carbon-based chain of at least 4 carbon atoms. Natural fatty acids have a carbon-based chain of 4 to 28 carbon atoms (generally an even number). The term “long-chain fatty acid” is used for a length of 14 to 22 carbons and the term “very-long-chain fatty acid” is used if there are more than 22 carbons. Conversely, the term “short-chain fatty acid” is used for a length of 4 to 10 carbons, especially 6 to 10 carbon atoms, in particular 8 or 10 carbon atoms. Those skilled in the art know the associated nomenclature and in particular use:
Ci−Cp to denote a range of Ci to Cp fatty acids,
Ci+Cp, the total of the Ci fatty acids and of the Cp fatty acids.

For example:
the fatty acids having 14 to 18 carbon atoms are written as “C14-C18 fatty acids”,
the total of the C16 fatty acids and of the C18 fatty acids is written as C16+C18;
for a saturated fatty acid, a person skilled in the art will use the following nomenclature Ci: 0, wherein i is the number of carbon atoms of the fatty acid. Palmitic acid will for example be denoted by the nomenclature (C16:0);
for an unsaturated fatty acid, a person skilled in the art will use the following nomenclature Ci: x n-N where N will be the position of the double bond in the unsaturated fatty acid starting from the carbon opposite the acid group, i is the number of carbon atoms of the fatty acid, and x is the number of double bonds (unsaturations) of this fatty acid. Oleic acid will for example be denoted by the nomenclature (C18:1 n-9).

Advantageously, the iodized oil according to the invention comprises or consists of derivatives of iodized fatty acids, preferentially of ethyl esters of iodized fatty acids, more preferentially of ethyl esters of iodized fatty acids of poppy oil, of olive oil, of rapeseed oil, of peanut oil, of soybean oil or of walnut oil, even more preferentially of ethyl esters of iodized fatty acids of poppy oil or of olive oil. More preferentially, the iodized oil according to the invention comprises or consists of ethyl esters of iodized fatty acids of poppy oil (said poppy also being known as blue seeded opium poppy or Papaver somniferum var. nigrum). The poppy oil, also known as poppyseed oil, preferentially contains more than 80% of unsaturated fatty acids (in particular of linoleic acid (C18:2 n-6) and of oleic acid (C18:1 n-9)) of which at least 70% of linoleic acid and at least 10% of oleic acid. The iodized oil is obtained from complete iodization of an oil such as poppy oil under conditions which allow bonding of one iodine atom for each double bond of the unsaturated fatty acids (Wolff et al. 2001, Medicine 80, 20-36) followed by trans-esterification.

The iodized oil according to the invention preferentially contains from 29% to 53% (w/w), more preferentially 37% to 39% (w/w), of iodine.

As examples of iodized oils, mention may be made of Lipiodol®, Brassiodol® (derived from rapeseed (Brassica compestis) oil), Yodiol® (derived from peanut oil), Oriodol® (derived from poppy oil but in the form of fatty acid triglycerides) and Duroliopaque® (derived from olive oil).

Preferentially, the iodized oil is Lipiodol®, which is an iodized oil used as a contrast product and in certain interventional radiology procedures. This oil is a mixture of ethyl esters of iodized and non-iodized fatty acids of poppyseed oil. It consists mainly (in particular, of more than 84%) of a mixture of ethyl esters of long-chain iodized fatty acids (in particular C18 fatty acids) derived from poppyseed oil, preferentially of a mixture of ethyl monoiodostearate and ethyl diiodostearate. The iodized oil may also be an oil based on a monoiodized ethyl ester of stearic acid (C18:0) derived from olive oil. A product of this type, called Duroliopaque® was sold a few years ago.

The main characteristics of Lipiodol® are the following:

| Compounds | Proportions in the fatty acid mixture |
|---|---|
| Ethyl palmitate (Ethyl C16:0) | 4.6% to 6.7% (w/w), |
| | preferentially 4.8% (w/w) |
| Ethyl stearate (Ethyl C18:0) | 0.8% to 1.9% (w/w), |
| | preferentially 1.2% (w/w) |
| Ethyl monoiodostearate | 11.3% to 15.3% (w/w), |
| | preferentially 13.4% (w/w) |
| Ethyl diiodostearate | 73.5% to 82.8% (w/w), |
| | preferentially 78.5% (w/w) |

Other characteristics of Lipiodol ®:

| | |
|---|---|
| Iodine | 37% to 39% (w/w) (i.e. 480 mg/ml) |
| Viscosity | |
| at 37° C. | 25 mPa · s |
| at 20° C. | 50 mPa · s |
| Density | 1.268-1.290 g/cm³ at 20° C., preferentially 1.28 |

Preferentially, the amount of iodized oil present in the composition according to the invention does not exceed 15 ml.

Preferentially, the lipid phase consists essentially of iodized oil as defined above and of a surfactant of formula (I) or (I′). In one particular embodiment of the invention, the lipid phase consists essentially of iodized oil as defined above, of a non-iodized oil as defined above and of a surfactant of formula (I) or (I′).

### Anti-Cancer Agent

The anti-cancer agent vectorized by the composition according to the invention or included in the composition in the form of an emulsion according to the invention is preferentially chosen from anthracyclines, platinum complexes, anthracycline-related compounds such as mitoxantrone and nemorubicin, antibiotics such as mitomycin C (Ametycine®), bleomycin and actinomycin D, other antineoplastic compounds such as irinotecan, 5-fluorouracil (Adrucil®), sorafenib (Nevaxar®), sunitinib (Sutent®), regorafenib, brivanib, orantinib, linsitinib, erlotinib, cabozantinib, foretinib, tivantinib, fotemustine, tauromustine (TCNU), carmustine, cytosine C, cyclophosphonamide, cytosine arabinoside (or cytarabine), paclitaxel, docetaxel, methotrexate, everolimus (Afinitor®), PEG-arginine deiminase, the tegafur/gimeracil/oteracil combination (Teysuno®), muparfostat, peretinoin, gemcitabine, bevacizumab (Avastin®), ramucirumab, floxuridine, immunostimulants such as GM-CSF (granulocyte-macrophage colony-stimulating factor) and recombinant forms thereof: molgramostim or sargramostim (Leukine®), OK-432 (Picibanil®), interleukin-2, interleukin-4 and tumor necrosis factor-alpha (TNFalpha), ¹²⁵I-labeled anti-CEA (carcinoembryonic antigen) antibodies, microspheres loaded with one of the abovementioned compounds, radioelements, complexes of said radioelements with chelates, magnetic particles based on an iron compound (ultrasmall superparamagnetic particles of iron oxide or USPIOs) and/or on a gadolinium chelate, radioactive microspheres, nucleic acid sequences or a mixture of one or more of these compounds (preferentially a mixture of one or more anthracyclines or a mixture of an anthracycline and a radioelement, as mentioned above, or a mixture of an anthracycline and a particle based on an iron compound and/or on a gadolinium chelate.

Preferentially, the aqueous phase of the composition according to the invention comprises 0.5% to 2.5% (w/v), more preferentially 1% to 2% (w/v), of anti-cancer agent in the aqueous phase.

The composition in emulsion form may comprise one or more anti-cancer agents. Preferably, at least one anti-cancer agent is water-soluble, i.e. it is more than 50% soluble in the aqueous phase. Thus, when the composition in emulsion form comprises only one anti-cancer agent, said agent is preferably water-soluble and is therefore in the dispersed aqueous phase. When the composition in emulsion form comprises several anti-cancer agents, some of them may be in the continuous lipid phase.

The preferential anti-cancer agent is chosen from anthracyclines, mitomycin C, platinum complexes, radioelements and the complexes thereof listed above. The anti-cancer agent is more preferentially chosen from anthracyclines and even more preferentially from doxorubicin, epirubicin, nemorubicin and idarubicin.

Advantageously, the anti-cancer agent is chosen from intercalating agents such as doxorubicin, epirubicin, idarubicin, nemorubicin, mitoxantrone and pirarubicin; alkylating agents such as cisplatin, carboplatin, oxaliplatin, lobaplatin, cyclophosphonamide and mitomycin C, fotemustine; topoisomerase type 1 inhibitors such as irinotecan; topoisomerase type 2 inhibitors such as doxorubicin and mitoxantrone; tyrosine kinase inhibitors such as everolimus; multikinase inhibitors such as sorafenib, antimetabolite agents such as 5-fluorouracil, methotrexate and gemcitabine, the radioelements as listed above, complexes of these radioelements with macrocyclic chelates, magnetic particles based on an iron compound, radioactive microspheres, nucleic acid sequences and a mixture thereof.

Preferentially, the anthracyclines mentioned above are chosen from doxorubicin (or adriamycin sold under the name Adriblastine® by Pfizer), epirubicin (Farmorubicin®), idarubicin (Zavedos®), daunorubicin, pirarubicin, nemorubicin and a mixture of one or more of these compounds.

Preferentially, the platinum complexes mentioned above are chosen from cisplatin (Platinol AQ®), carboplatin, miriplatin, oxaliplatin (Eloxatine®), lobaplatin and a mixture of one or more of these compounds.

Preferentially, the radioelements mentioned above are chosen from rhenium 186 (186Re), rhenium 188 (¹⁸⁸Re), yttrium 90 (⁹⁰Y), lutetium 177 (¹⁷⁷Lu), holmium 166 (¹⁶⁶Ho), iodine 125 (125I), (131, iodine ¹³¹I), phosphorus 32 (³²P), strontium 89 (⁸⁹Sr), samarium 153 (¹⁵³Sm), copper 67 (⁶⁷Cu), tin 117m (^{117m}Sn) bismuth 213 (²¹³Bi), bismuth 212 (²¹²Bi), astate 211 (²¹¹At), radium 223 (²²³Ra), indium 111 ¹¹¹In), gallium 67 (⁶⁷Ga), gallium 68 (⁶⁸Ga), metastable technetium 99 (99mTc) and a mixture of one or more of these compounds. The radioelement, optionally in a form complexed with linear or macrocyclic chelates, is preferentially chosen from in, ¹⁸⁸Re, ⁹⁰Y, ¹⁷⁷Lu, ¹⁶⁶Ho, ¹³¹I, ¹¹¹In, ⁶⁷Ga, ⁶⁸Ga and 99mTc or even more preferentially from ¹⁸⁸Re, ⁹⁰Y, ¹⁷⁷Lu, ¹⁶⁶Ho and ¹³¹I. Preferentially, the chelates of the complexes of these radioelements mentioned above are chosen from linear chelates and macrocyclic chelates such as DOTA, PCTA, DTPA, NOTA, and derivatives thereof, more preferentially from macrocyclic chelates such as DOTA, PCTA, NOTA, and derivatives thereof. Yttrium 90 (⁹⁰Y) and the complexes of yttrium 90 and of macrocyclic chelates as defined above are preferential compounds in their respective categories.

Preferentially, the nucleic acid sequences mentioned above are chosen from deoxyribonucleic acid (DNA) and ribonucleic acid (RNA) sequences, more preferentially chosen from DNA or RNA sequences vectorized by gene therapy vectors, such as viral vectors chosen from adenovirus (DNA virus) vectors, retrovirus (RNA virus) vectors, vectors derived from adeno-associated viruses or AAVs and vectors derived from other viruses (such as Herpes Simplex viruses (HSVs), poxviruses, influenza viruses), and nonviral vectors such as polycations or nanoparticles (in particular of hydroxyapatite or modified hydroxyapatite (such as poly-L-lysine (PLL)-modified hydroxyapatite)) and interfering RNA (siRNA for small interfering RNA) or double-stranded RNA (dsRNA) sequences.

The nucleic acid sequences are preferentially chosen from the native or modified sequences or a part of the native or modified sequences of the gene encoding the p53 protein, encoding the Rb protein (in particular the Rb1 gene) or encoding the gene encoding interleukin 12 (IL-12), or the respective transcripts thereof (i.e. in RNA form).

The commercial form of these anti-cancer agents is usually the lyophilized form or the pulverized form (i.e. in powder form). These lyophilisates or powders of anti-cancer agents may contain the excipients conventionally used in the pharmaceutical field: lactose (dissolving and lyophilizing agent), methyl para-hydroxybenzoate (antioxidant) and/or sodium chloride (NaCl).

For the purposes of the present description, the term “particles based on an iron compound” is intended to mean particles comprising or consisting of an iron compound, generally comprising iron (III), generally an iron oxide or hydroxide. The term ultra small particles of iron oxide or USPIOs is often used.

As a general rule, the magnetic particles are totally or partly composed of iron hydroxide; of iron oxide hydrate; of ferrites; of mixed iron oxides such as mixed iron oxides of cobalt, of nickel, of manganese, of beryllium, of magnesium, of calcium, of barium, of strontium, of copper, of zinc or of platinum; or a mixture thereof.

According to one particularly preferred variant, the magnetic particles are superparamagnetic.

The magnetic particles before being covered with the appropriate coating, then preferably have a crystal diameter of from 5 to 200 nm, even better still from 10 to 60 nm or from 10 to 20 nm.

In one advantageous embodiment, the magnetic particles based on an iron compound are covered with a hydrophilic compound, preferentially of polyethylene glycol (PEG) type, more preferentially a PEG having a molar mass included from 1500 to 3000.

In another advantageous embodiment, the magnetic particles based on an iron compound are covered with an unsaturated, preferentially monounsaturated, fatty acid, even more preferentially with oleic acid (C18:1 n-9). The magnetic particles thus made liposoluble are suspended in the continuous lipid phase.

For the purposes of the present application, the term “ferrite” denotes iron oxides of general formula [x Fe₂O₃, y MO_{z}], wherein M denotes a metal that can be magnetized under the effect of a magnetic field, such as Fe, Co, Ru, Mg or Mn, it being possible for the magnetizable metal to be optionally radioactive.

Preferentially, the magnetic particles of the compositions of the invention comprise a ferrite, in particular maghemite (γ Fe₂O₃) or magnetite (Fe₃O₄), or else mixed ferrites of cobalt (Fe₂CoO₄) or of manganese (Fe₂MnO₄). In this context, preference is most particularly given to the magnetic particles totally or partly composed of a ferrite, and preferably essentially (i.e. more than 90%, preferentially more than 95%, even more preferentially more than 98% by weight), of maghemite or of magnetite or of a mixture thereof.

Preferentially, the radioactive microspheres mentioned above consists of a cation exchange resin (comprising for example a polyvinyl alcohol or a copolymer comprising styrene and divinylbenzene, such as Aminex 50W-X4 from the company Biorad) labeled with yttrium 90 (SIR-Spheres® sold by the company SIRTeX Medical Ltd) or consists of glass into which yttrium 90 has been incorporated (TheraSphere® sold by the company BTG) or consists of a polymer such as polylactic acid (PLLA) and of one of the radioelements mentioned above, holmium (166Ho) then being the preferred radioelement. More preferentially, it is yttrium in the form of ⁸⁹Y₂O₃ which is incorporated into the microspheres consisting of glass, said microspheres then being irradiated with neutrons in order to make them radioactive by converting the cold yttrium ⁸⁹Y to radioactive yttrium ⁹⁰Y. Even more preferentially, the microspheres consisting of a cation exchange resin or consisting of glass have respectively a diameter of from 20 to 60 μm and from 20 to 30 μm. The microspheres of the SIR-Spheres type were in particular the subject of patent EP 0 740 581 B1.

Preferentially, the microspheres loaded with one of the compounds mentioned above are loaded with an anthracycline such as doxorubicin, epirubicin or idarubicin or with a topoisomerase type I inhibitor such as irinotecan or with a platinum complex such as cisplatin. These microspheres are preferentially produced from polyvinyl alcohol (PVA). Preferentially, they consist of a hydrogel of PVA and more preferentially consist of a polymer of PVA modified with sulfonate SO₃⁻ groups to which the compounds mentioned above attach when they are positively charged (DC Beads®, DC-Beads M1® and LC-Beads® sold by the company Biocompatibles) or they are produced from monomers such as vinyl acetate and methyl acrylate which, when they are combined together, form a PVA/acrylic copolymer (copolymer of poly(sodium acrylate-co-vinyl alcohol)) modified with carboxylate COO— groups to which the compounds mentioned above attach, by simple ionic bonding, when they are positively charged (Hepasphere® or Quadrasphere® sold by Merit Medical). These microspheres can also consist of a polyphosphazene polymer and are then loaded with doxorubin, with epirubicin, with idarubicin or with irinotecan (Embozene Tandem® microspheres sold by the company Celonova Biosciences). They can also consist of a polymer obtained from hydrolyzed potato flour crosslinked and substituted with glycerol ether groups, and are then loaded with doxorubicin, actinomycin D, tauromustine, cisplatin, carboplatin, mitomycin C, fotemustine, carmustine, irinotecan, 5-FU, floxuridine or docetaxel, with ¹²⁵I-labeled anti-CEA (carcinoembryonic antigen) antibodies or with 99mTc-DTPA complex (Embocept® S microspheres sold by Pharmacept).

### Surfactant

It is recalled that the term “surfactant” refers to a composition with an amphiphilic structure which confers thereon a particular affinity for interfaces of water/oil type, thereby giving it the ability to lower the free energy of these interfaces and to stabilize dispersed systems.

The composition according to the invention comprises at least one surfactant of formula (I) or (I′) as defined above. It can therefore comprise a surfactant of formula (I) or (I′) or a mixture of surfactants of formula (I) or (I′).

The surfactant has the formula (I) or (I′) as defined above, preferably in which s is 0 or 1, m represents an integer from 2 to 10 and R₁ represents a group of formula (II) as defined above, in which n represents an integer from 5 to 7, o represents an integer from 1 to 3, p represents an integer from 3 to 5, q represents an integer from 2 to 5 and r is 0 or 1. Even more preferentially, in formula (I) or (I′) as defined above, s is 1, m represents an integer from 2 to 5 and n is 7, o is 1, p is 5 and q represents an integer from 2 to 4 and r is 1 in formula (II) represented by R₁.

The HLB (meaning hydrophilic-lipophilic balance) is a magnitude, well known to those skilled in the art, characteristic of a surfactant. Preferentially, the surfactant according to the invention is a surfactant with a low HLB, i.e. a surfactant having an HLB value included from 1 to 8, preferentially included from 1 to 6. The HLB makes it possible to determine the type of oil-in-water or water-in-oil emulsion, as illustrated in the article by W. C. Griffin (“Classification of Surface-active agents by “HLB””, Journal of the Society of Cosmetic Chemists, 1949, 311-326). This article indicates in particular that, for surfactants of which the HLBs are from 4 to 6, emulsions of W/O type are observed, while for surfactants of which the HLBs are from 8 to 18, emulsions of O/W type are instead observed.

Advantageously, the surfactant of formula (I) or (I′) is soluble in the iodized oil, in particular in the proportion ranges indicated above.

Advantageously, the surfactant of formula (I) or (I′) according to the invention is chosen from polyglyceryl polyricinoleate and PEG-30 dipolyhydroxystearate.

Polyglyceryl polyricinoleate or PGPR (Palsgaard®4125, Palsgaard®4150, Palsgaard®4110, Palsgaard®4120 or Palsgaard®4175) is a surfactant which has, as hydrophilic group, polyglycerol (preferably consisting of at least 75% of di- and triglycerol and of at most 10% of heptaglycerol) and, as hydrophobic group, interesterified ricinoleiques fatty acids. It has an HLB of 1.5.

It corresponds to a surfactant of formula I, as defined above, in which:
s is 1,
m represents an integer from 2 to 5,
R₁ represents a group of formula (II) as defined above, in which n is 7, o is 1, p is 5, q is 2 to 4 and r is 1,
R₂ represents R₁ and/or a hydrogen atom.

Preferentially, the surfactant of formula (I) or (I′) is a mixture of surfactants of formula (I) or (I′) in which:
s is 1,
m is 2, 3, 4 or 5,
R₁ represents a group of formula (II) as defined above, in which n is 7, o is 1, p is 5, q is 2, 3 or 4 and r is 1,
R₂ represents R₁ and/or a hydrogen atom.

Preferentially, the surfactant of formula (I) or (I′) according to the invention is a mixture of surfactants, chosen from the compounds of formula:

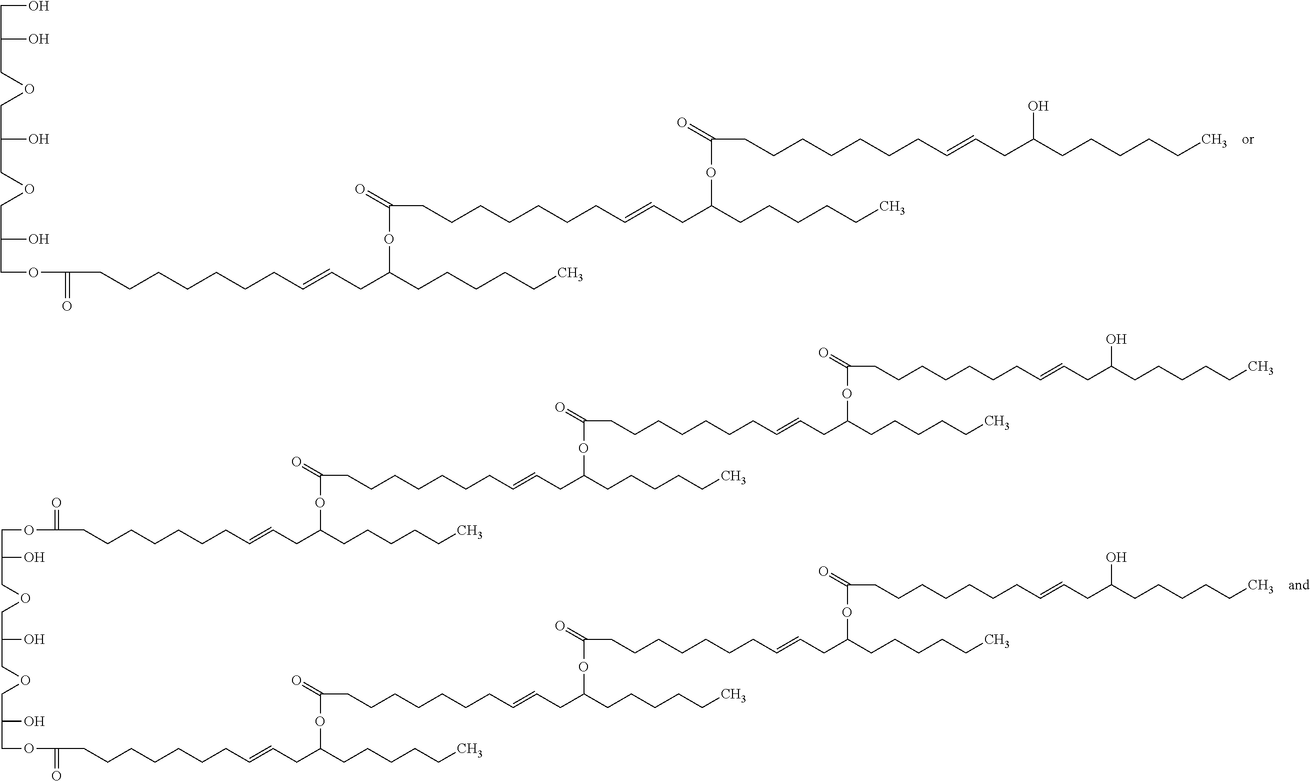 [SMILES: CCCCCCC(O)C/C=C/CCCCCCCC(=O)OC(C/C=C/CCCCCCCC(=O)OC(C/C=C/CCCCCCCC(=O)OCC(O)COCC(O)COCC(O)CO)CCCCCC)CCCCCC ; CCCCCCC(O)C/C=C/CCCCCCCC(=O)OC(C/C=C/CCCCCCCC(=O)OC(C/C=C/CCCCCCCC(=O)OC(C/C=C/CCCCCCCC(=O)OCC(O)COCC(O)COCC(O)COC(=O)CCCCCCC/C=C/CC(CCCCCC)OC(=O)CCCCCCC/C=C/CC(CCCCCC)OC(=O)CCCCCCC/C=C/CC(CCCCCC)OC(=O)CCCCCCC/C=C/CC(O)CCCCCC)CCCCCC)CCCCCC)CCCCCC]

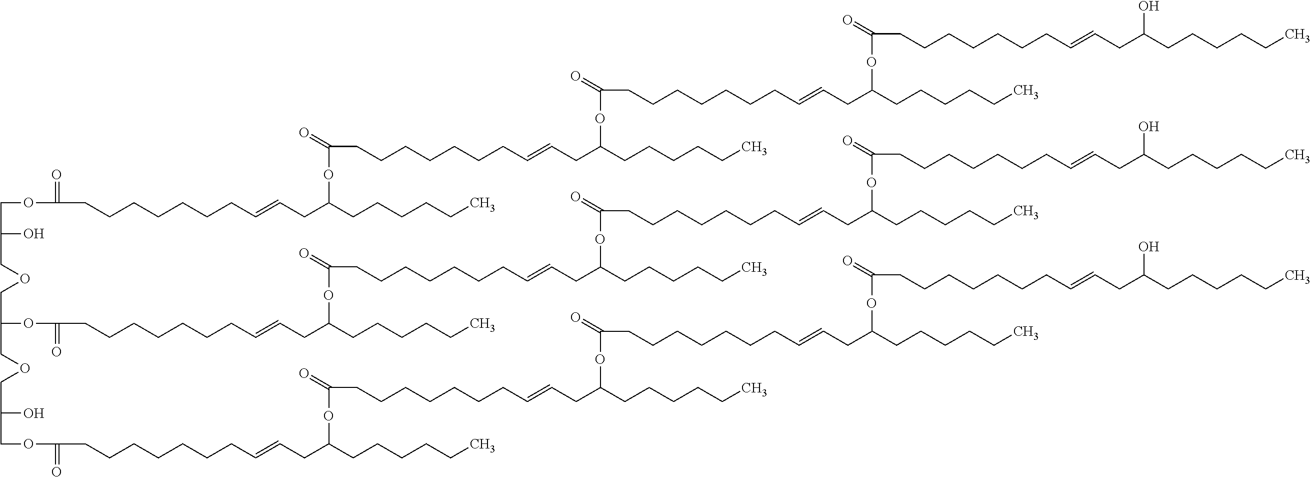 [SMILES: CCCCCCC(O)C/C=C/CCCCCCCC(=O)OC(C/C=C/CCCCCCCC(=O)OC(C/C=C/CCCCCCCC(=O)OC(C/C=C/CCCCCCCC(=O)OCC(O)COCC(COCC(O)COC(=O)CCCCCCC/C=C/CC(CCCCCC)OC(=O)CCCCCCC/C=C/CC(CCCCCC)OC(=O)CCCCCCC/C=C/CC(CCCCCC)OC(=O)CCCCCCC/C=C/CC(O)CCCCCC)OC(=O)CCCCCCC/C=C/CC(CCCCCC)OC(=O)CCCCCCC/C=C/CC(CCCCCC)OC(=O)CCCCCCC/C=C/CC(CCCCCC)OC(=O)CCCCCCC/C=C/CC(O)CCCCCC)CCCCCC)CCCCCC)CCCCCC]

PEG-30 dipolyhydroxystearate (Cithrol® DPHS and formerly Arlacel® P135 sold by the company Croda) as an HLB of 5-6. The name PEG is in accordance with the nomenclature conventions set by the INCI, the value 30 specified above corresponding to the average number of ethylene oxide monomer units.

It corresponds to a surfactant of formula 1, as defined above, in which:
s is 0,
m is 30,
R₁ represents a group of formula (II) as defined above, in which n is 9, o is 1, p is 5, q is 7 and r is 0,
R₂ is identical to R₁.

### Use of the Composition According to the Invention

According to a second subject, the invention relates to the use of the composition as defined above, for vectorizing an anti-cancer agent. The invention also relates to a composition in emulsion form as defined above, for use thereof in the treatment of cancer or metastases thereof, preferentially by transarterial chemoembolization. In one advantageous embodiment, the invention relates to the use of a composition according to the invention, for preparing a medicament for treating cancer or metastases thereof, preferentially by transarterial chemoembolization. Transarterial chemoembolization is defined as the transarterial percutaneous introduction of a substance in order to obstruct a blood vessel in combination with an anti-cancer agent, in order to deliver a therapeutically effective amount of this agent into a tumor. Preferentially, the cancer thus treated is chosen from liver cancer (in particular primary liver cancer, such as hepatocellular carcinoma or HCC), cholangiocarninoma, hepatic metastases of primary cancers chosen from colorectal cancer, neuroendocrine tumors, breast cancers, kidney cancers and melanomas.

The chemoembolization of an hepatic tumor is preferentially carried out by implementing the following successive steps:

a) percutaneous catheterization from the femoral artery,

b) administration of the emulsion according to the invention until stasis is observed in the second-order or third-order branches,

c) optionally, administration of an embolizing agent in the tumor after the emulsion has been administered.

Preferentially, the emulsion according to the invention thus administered comprises no more than 20 ml of iodized oil, more preferentially no more than 15 ml of iodized oil.

The catheterization, which consists in bringing a tube, called a catheter, into the hepatic artery and then into the branch of this artery which perfuses the cancerous lesion, is advantageously carried out with the assistance of an imaging technique. Interventional radiologists are moreover provided with guidance software in order to enable them to place their catheter as optimally as possible.

The term “embolizing agent” is intended to mean one or more compounds which make it possible to slow down or stop, definitively or temporary, the blood flow in a vessel. As examples of an “embolizing agent”, mention may be made of the gelatin sponge, gelatin foam particles (Gelfoam®, Spongel®, Curaspon®), polyvinyl alcohol (PVA) or calibrated microspheres based, for example, on trisacrylgelatin, on PVA (Ivalon®, Contour®), etc.

Advantageously, before the chemoembolization procedure, an angiography or arteriography, carried out using an angioscan or an MR angio (magnetic resonance angiography or MRA), and usually an injection of contrast product (for example, for the angioscan: water-soluble iodinated contrast products such as iobitridol (Xenetix®) or iohexol (Omnipaque®), and for the MR angio: gadolinium chelates such as gadoteric acid (Dotarem®) or gadobutrol (Gadovist®)), is performed in order to pinpoint the visceral vascularization and the arterial perfusion of the tumor(s).

This chemoembolization technique can be used alone or in combination with one or more other techniques mentioned below. It can also be replaced with one of these other techniques.

When the anti-cancer agent is chosen from radioelements or complexes of radioelements with macrocyclic chelates mentioned above, the technique used is internal selective radiotherapy or radioembolization. It consists in injecting the composition according to the invention directly into the branch of the hepatic artery which perfuses the tumor. This technique has the advantage of delivering a very significant irradiation to the tumor without, however, significantly irradiating the healthy liver and the other organs of the patient.

When the anti-cancer agent is chosen from magnetic particles based on an iron compound (USPIOs), the technique used is magnetic hyperthermia ablation. This consists in inducing a local increase in temperature at the level of the tumor tissue, the tumor cells being more sensitive to an increase in temperature than healthy cells. This increase in temperature is caused by using an external stimulus and in particular the application of an alternating magnetic field to the area that it is desired to treat. Two types of hyperthermia are distinguished depending on the temperature reached: for temperatures above 46° C., it is possible to induce tissue necrosis and the term thermoablation is then used; temperatures of 42° C. to 46° C. modify the functions of numerous structural and enzymatic proteins, modifying cell development and differentiation and possibly inducing apoptosis, the term moderate hyperthermia is then used. If the cells do not die, they become more sensitive to ionizing radiation or to chemotherapy.

When the anti-cancer agent is a nucleic acid sequence chosen from sequences of deoxyribonucleic acid (DNA) and ribonucleic acid (RNA) vectorized by a viral vector or a nonviral vector as mentioned above or an interfering RNA (siRNA for small interfering RNA) or double-stranded RNA (dsRNA) sequence, the technique used is gene therapy, sometimes also called “genotherapy”. The principle of this approach is to introduce a foreign gene of which the expression product induces (directly or indirectly) the death of the tumor cells. Schematically, three approaches can be used: a) the induction of an immune defense (“immunostimulation”) by modifying the tumor cell membrane antigens; b) the transfer of a tumor “suppressor” gene into the genome of the tumor cell, or finally c) the transfer of a “suicide” gene which makes it possible to convert a nonactive anti-cancer agent prodrug into a molecule that is toxic to the tumor cells.

All these various techniques are known to those skilled in the art. The latter will know how to easily chose the parameters to be adjusted in order to carry out these techniques using the composition according to the invention.

Unless otherwise indicated, the terms “treating” and “treatment” are intended to mean any action aimed at improving the comfort, well-being and survival of an individual, this term therefore covering both attenuating, decreasing, relieving and curing.

### Preparation of the Composition in the Form of an Emulsion According to the Invention

The composition in emulsion form is preferentially prepared extemporaneously.

The invention also relates to a method for preparing a composition in emulsion form as defined above, comprising the following steps:

a) mixing the surfactant of formula (I) or (I′) as defined above in the iodized oil, and

b) mixing the solution obtained in step a) with an aqueous solution comprising an anti-cancer agent.

The aqueous solution mixed with the solution obtained in step a) can also comprise a densifying agent as defined above.

The lipid phase prepared in step a) can also comprise a non-iodized oil as defined above.

The mixing carried out in step b) can be carried out by any means known to those skilled in the art. Preferentially, a three-way tap is used. The iodized oil comprising the surfactant is placed in a first syringe which is attached to the three-way tap. The aqueous solution comprising the anti-cancer agent is placed in a second syringe which is also attached to this three-way tap at 90°.

Mixing of the two phases is carried out by alternately pushing on the plungers of the two syringes (preferentially from 20 to 35 times). Preferentially, all of the mixture is passed through one syringe and then through the other every 1 to 2 seconds. The third channel of the tap makes it possible to attach a catheter which is selectively advanced, under fluoroscopic control, as far as the tumor lesion, for administration of the emulsion.

Preferentially, the preparation of the composition according to the invention is carried out at a temperature between 10 and 40° C., more preferentially between 20 and 30° C.

### Marketing Forms for the Composition According to the Invention

The invention also relates to a kit comprising:
a surfactant of formula (I) or (I′) as defined above,
an iodized oil,
an anti-cancer agent,

as combination products for use simultaneously, separately or spread out over time, for use thereof for treating cancer.

The surfactant, the iodized oil and the anti-cancer agent (generally dissolved in an aqueous solution) are in three different containers. Generally, the mixing of the [surfactant/iodized oil/anti-cancer agent in aqueous solution] results in the composition in the form of an emulsion according to the invention.

Furthermore, the invention relates to a kit comprising:
a composition comprising the surfactant of formula (I) or (I′) as defined above and an iodized oil,
an anti-cancer agent,

as combination products for use simultaneously, separately or spread out over time, for use thereof for treating cancer.

The composition and the anti-cancer agent (preferentially provided in lyophilized form) are in two different containers. Preferentially, the anti-cancer agent is dissolved in an aqueous solution extemporaneously or the day before the procedure. Preferably, the composition consists of a mixture of surfactant of formula (I) or (I′), of an iodized oil and optionally of a non-iodized oil. Generally, the mixing of the composition and of the anti-cancer agent in aqueous solution results in the composition in the form of an emulsion according to the invention.

The invention also relates to the use of a kit comprising:
a surfactant of formula (I) or (I′) as defined above,
an iodized oil,

as combination products for vectorizing an anti-cancer agent. The surfactant and the iodized oil are in two different containers.

The invention also relates to the use of a composition comprising:
a surfactant of formula (I) or (I′) as defined above dissolved in an iodized oil,

as a product for vectorizing an anti-cancer agent. The surfactant is dissolved in the iodized oil in the same container.

The term “container” is intended to denote any pharmaceutically acceptable receptacle which can contain a product. By way of example, mention may be made of an ampoule, a bottle or a prefilled syringe.

The term “pharmaceutically acceptable receptacle” is intended to denote any receptacle which does not interact with the product, preferentially any receptacle which does not release compounds into the iodized oil and does not degrade the iodized oil.

The examples which appear hereinafter are presented by way of nonlimiting illustration of the invention.

FIG. 1: Plasma kinetics of doxorubicin in rats carrying a hepatocellular carcinoma after injection by a chemoembolization (TACE) procedure of several emulsions.

FIG. 2: Plasma kinetics of doxorubicin in rabbits carrying a hepatocellular carcinoma after injection by a chemoembolization (TACE) procedure of several emulsions.

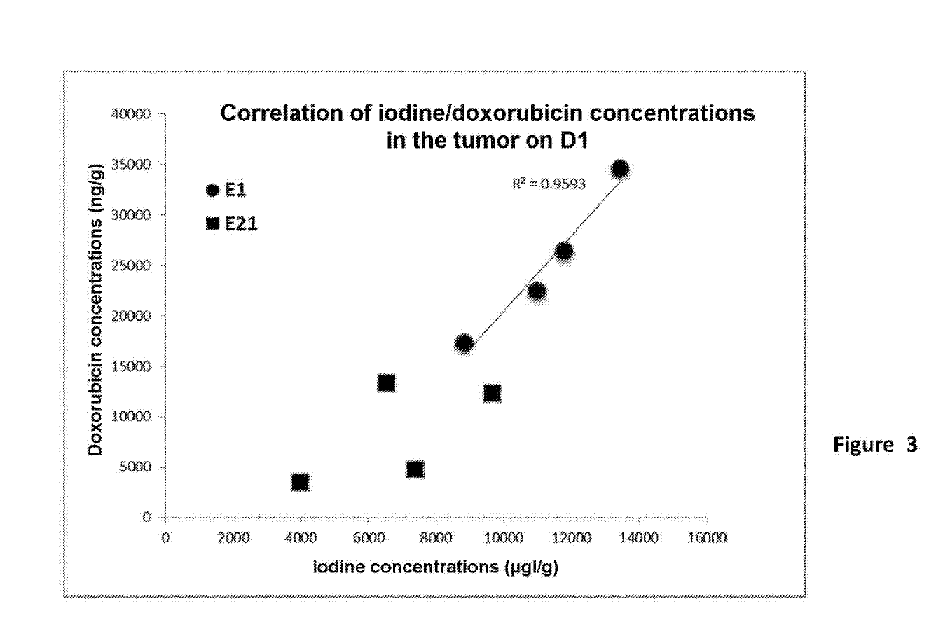

FIG. 3: Results of a study of correlation between the amount of Lipiodol® measured in a tumor and the amount of doxorubicin measured in said tumor according to the emulsion administered.

### EXAMPLE 1

### 1. Preparation of Compositions in the Form of an Emulsion According to the Invention

1.1. Emulsions of Lipiodol® and of Anthracycline

50 mg of doxorubicin (Adriblastina®) were reconstituted in 2.5 ml of Xenetix® 250 (250 mg of iodine/ml). After manual stirring for 30 seconds for good dissolution, the solution obtained was removed with a 20 ml luer lock syringe. This syringe was then placed on a three-way tap.

PGPR (1% w/v total, 100 mg-Interchim) was dissolved in 7.5 ml of Lipiodol® by manual stirring.

The oil obtained was removed with a 20 ml luer lock syringe, which was also placed on the three-way tap at 90° C. 34 passes, i.e. 17 back-and-forward motions, at medium force were carried out, beginning with the water into the oil.

For these emulsions, the volumes of the aqueous phase and of the lipid phase chosen were respectively 2.5 ml (i.e. 25% v/v) and 7.5 ml (i.e. 75% v/v). The aqueous phase/lipid phase ratio was ⅓.

Other emulsions were prepared:
by replacing the doxorubicin as anti-cancer agent with idarubicin (Zavedos®), mitomycin C (Kyowa) or epirubicin (Farmorubicine®), and/or
by introducing no densifying agent, or
by replacing the densifying agent Xenetix® 250 with Xenetix® 300 (300 mg of iodine/ml), Iopamiron® 350, Iopamiron® 300, Iomeron® 300, Ultravist® 300 or Omnipaque® 240, or
by replacing the surfactant PGPR with Cithrol™ DPHS (PEG-30 dipolyhydroxystearate), or
by changing the proportion of surfactant.

For the Cithrol™ DPHS, dissolution was obtained by using ultrasound (Vial tweeter, 3×45 s).

### Verification of the Sense of the Emulsion:

Once the emulsion had been prepared, the sense thereof was verified by means of a simple visual test. Two bottles were prepared: one with aqueous phase (Xenetix® 250 or Xenetix® 300 as appropriate) and the other with iodized oil (Lipiodol®).

A drop of freshly prepared emulsion was added to each of the two bottles. The drop dispersed in the bottle of Lipiodol® and did not disperse in the bottle of Xenetix®; the emulsion was therefore indeed a W/O (water-in-oil) emulsion.

The red doxorubicin droplets were clearly visible in a yellow background of oil. The size of the aqueous phase droplets was evaluated using an optical microscope.

The principal emulsions prepared are described in the following table:

| | | | | | Sizes of the | |
|---|---|---|---|---|---|---|
| | | Proportion of | Nature of the | Nature of the | aqueous | Visual |
| Product | Nature of the | surfactant used | anti-cancer | densifying | phase | stability |
| number | surfactant used | (% w/v) | agent used | agent used | droplets | observed* |
| E1 | PGPR | 1% | Doxorubicin | Iobitridol** | 5-20 μm | No phase |
| | | | | | | separation |
| | | | | | | at 24 h |
| E2 | PGPR | 1% | Doxorubicin | None | 5-40 μm | Phase |
| | | | | | | separa- |
| | | | | | | tion <5% |
| | | | | | | at 24 h |
| E3 | PGPR | 1% | Doxorubicin | Iobitridol*** | 5-20 μm | No phase |
| | | | | | | separation |
| | | | | | | at 24 h |
| E4 | PGPR | 1% | Doxorubicin | Iopamidol**** | 5-10 μm | No phase |
| E5 | PGPR | 0.5% | Doxorubicin | Iobitridol** | 5-20 μm | separation |
| E6 | PGPR | 0.3% | Doxorubicin | Iobitridol** | 5-20 μm | at 24 h |
| E7 | PGPR | 1% | Mitomycin C | Iobitridol** | 5-10 μm | No phase |
| E8 | PGPR | 1% | Epirubicin | Iobitridol** | 5-20 μm | separation |
| E9 | PGPR | 0.7% | Idarubicin | Iobitridol** | 2-10 μm | at 24 h |
| E10 | Cithrol ™ | 1% | Doxorubicin | None | 5-20 μm | Phase |
| | DPHS | | | | | separa- |
| | | | | | | tion <5% |
| | | | | | | at 24 h |
| E11 | Cithrol ™ | 1% | Doxorubicin | Iobitridol** | 5-20 μm | No phase |
| | DPHS | | | | | separation |
| E12 | Cithrol ™ | 1% | Doxorubicin | Iobitridol*** | 5-20 μm | at 24 h |
| | DPHS | | | | | |

*at ambient temperature (20° C.)
**Xenetix ® 250
***Xenetix ® 300
****Iopamiron ® 250

These various emulsions prepared using a surfactant of formula (I) and various anti-cancer agents all demonstrated a stability in accordance with expectations.

1.2. Emulsions of Lipiodol® and of Radioelements

1.2.1. Emulsion of Lipiodol® and ⁹⁰YCl₃

A buffer solution (tris) was added to a radioactive solution of yttrium 90 in the form of an acid solution (yttrium chlorite, 0.05M HI) in order to bring the resulting solution to a pH compatible with use in the patient (6<pH<9). The solution obtained can be diluted in a volume of physiological saline so that the final volume does not exceed 10 ml. 1% (w/v) of PGPR was dissolved in 7.5 ml of Lipiodol® according to the technique described above. The lipid phase consisting of 7.5 ml of Lipiodol® and of the 1% of PGPR was then added to the aqueous phase and the emulsion was prepared by agitation by suctioning and resuspension of the suspension thus obtained.

1.2.2. Emulsion of Lipiodol® and of ⁹⁰YCl₃ Complexed with DOTA

The radioactive solution was added to a solution of DOTA (1,4,7,10-tetraazacyclododecane-N,N′,N″,N′″-tetraacetic acid) in a buffer at pH 6-7, and the medium was heated at 80° C. for 30 min.

A volume of physiological saline was added to this solution so that the final volume did not exceed 10 ml. 1% (w/v) of PGPR was dissolved in 7.5 ml of Lipiodol® according to the technique described above.

The lipid phase consisting of 7.5 ml of Lipiodol® and of the 1% of PGPR was then added to the aqueous phase and the emulsion was prepared by vigorous agitation of the suspension thus obtained.

1.3. Emulsion of Lipiodol® and of Iron-Based Magnetic Particles

### 1.3.1. Emulsion of Lipiodol®, of Iron-Based Magnetic Particles and of Anthracycline

The magnetic nanoparticles based on an iron compound, covered with oleic acid (synthesized according to the techniques known from the prior art) were totally dissolved in 60 g of Lipiodol® at 60° C. for 24 h. The total dissolution of said magnetic nanoparticles was assessed visually by noting the absence of aggregate visible to the naked eye. After a return to ambient temperature, the solution was stored or used to produce the emulsions.

A 50 mg bottle of Adriblastina® was reconstituted with 2.5 ml of Xenetix® 250. Stirring was carried out manually for 30 seconds for good dissolution. The solution obtained was removed with a 20 ml luer lock syringe, which was placed on a three-way tap.

PGPR (1% w/v total, 100 mg) was dispersed in 7.5 ml of Lipiodol® comprising the magnetic particles.

The oil obtained was removed with a 20 ml luer lock syringe, which was also placed on the three-way tap. 30 passes, i.e. 15 back-and-forward motions, at medium force were carried out beginning with the water into the oil.

### 1.3.2. Emulsion of Lipiodol® and of Iron-Based Magnetic Particles

The magnetic nanoparticles based on an iron compound (synthesized according to the techniques known from the prior art: see in particular WO 2004/058275), covered with a layer of gem-bisphosphonate of formula:

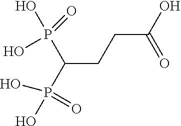 [SMILES: O=C(O)CCC(P(=O)(O)O)P(=O)(O)O]

coupled to PEG 2000, were totally dissolved in 10 g of physiological saline at 60° C. for 24 h so as to obtain a 0.5M iron concentration. The total dissolution of said magnetic nanoparticles was assessed visually by noting the absence of aggregate visible to the naked eye. After a return to ambient temperature, the solution was stored or used to produce emulsions.

2.5 ml of the solution obtained were removed with a 20 ml luer lock syringe, which was placed on a three-way tap. PGPR (1% w/v total, 100 mg) was dispersed in 7.5 ml of Lipiodol®. The oil obtained was removed with a 20 ml luer lock syringe, which was also placed on the three-way tap at 90°. 30 passes, i.e. 15 back-and-forward motions, at medium force were carried out beginning with the water into the oil.

### 2. Comparison with Emulsions not in Accordance with the Invention

Emulsions according to the same protocol as that specified in paragraph 1.1 or a slightly different protocol (the differences compared with the protocol of paragraph 1.1 are indicated in the table below: the respective volumes of the aqueous and lipid phases are calculated on the basis of their ratio) were prepared using either a concentration of PGPR not in accordance with the invention, or surfactants having a low or high HLB, such as surfactants of the Span® family (fatty acid esters of sorbitan), surfactants having a high HLB of the Cremophor® family (glycerol polyethylene glycol ricinoleate), of the Tween® family (polyoxyethylene fatty acid esters of sorbitan) or of the Pluronics® family (block copolymers based on ethylene oxides and propylene oxide, sold by BASF), and the Cithrol® PG32IS surfactant having a low HLB (HLB=6.7).

Densifying agents other than the nonionic iodinated contrast products were tested, such as PVP (polyvinylpyrrolidone), glycerol, or else dextran T40 (Sigma), but the maximum amounts that can be used do not make it possible to approach the density of an iodized oil such as Lipiodol®. Ioxaglic acid (Hexabrix®) does not make it possible to easily dissolve anti-cancer agents such as doxorubicin and considerably increases the osmolality of the composition.

The principal emulsions prepared are described in the following tables:

Emulsions Prepared with a Surfactant in Accordance with the Invention but Using a Concentration which does not Comply:

| | | | Nature of the | Aqueous | | |
|---|---|---|---|---|---|---|
| Product | Nature of the | Proportion of | anti-cancer | phase/lipid | | |
| number | surfactant used | surfactant used | agent used | phase ratio | Droplet sizes | Observations |
| E13 | PGPR | 0.2% by | Doxorubicin | 1/3 | Heterogeneity: | W/O |
| | | weight | 50 mg in 2.5 ml | | small drops | emulsion |
| | | relative to | of Xenetix ® 250 | | and coarser | Phase |
| | | the total | | | drops of | separation |
| | | volume of | | | 20-50 μm | |
| | | the emulsion | | | | |
| E13′ | PGPR | 0.2% by | Doxorubicin | 1/3 | Heterogeneity: | W/O |
| | | weight | 50 mg in 2.5 ml | | small drops | emulsion |
| | | relative to | of physiological | | and coarser | Phase |
| | | the total | saline | | drops of | separation |
| | | volume of | | | 20-50 μm | |
| | | the emulsion | | | | |
| E13″ | PGPR | 5% by | Epirubicin | 1/1 | Heterogeneity: | O/W |
| | | weight | 50 mg in 5 ml of | | drops ranging | emulsion |
| | | relative to | water | | from 5 to | total phase |
| | | the total | supplemented | | 50 μm | separation in |
| | | volume of | with 290 mg of | | | 2 hours |
| | | the emulsion | glucose | | | |
| | | | (5.8% w/v) | | | |

Emulsions Prepared with a Surfactant and/or a Densifying Agent not in Accordance with the Invention and/or in Aqueous Phase/Lipid Phase Ratios which do not Comply:

| | | Proportion | | | | | |
|---|---|---|---|---|---|---|---|
| | | of surfactant | | | | | |
| | | used by weight | | | | | |
| | | relative to the | Nature of the | Aqueous | | | |
| Product | Nature of the | total volume of | anti-cancer | phase/lipid | | | |
| number | surfactant used | the emulsion | agent used | phase ratio | Droplet sizes | | Observations |
| E14 | Span ® 80 | 1% (also | 2 ml of a | 1/4 or | Aggregates of | | W/O |
| | | tested: 0.5% | solution of | 1/3 | droplets of | | emulsion |
| | | and 0.8%) | doxorubicin | | 50-100 μm | | Phase |
| | | | 50 mg in | | | | separation |
| | | | 10 ml of | | | | whatever the |
| | | | Xenetix ® 250 | | | | aqueous |
| | | | | | | | phase/lipid |
| | | | | | | | phase ratio |
| | | | | | | | and the |
| | | | | | | | proportion of |
| | | | | | | | surfactant |
| | | | | | | | used |
| E15* | Span ® 80 | 1% | 2 ml of a | 1/4 | Heterogeneity: | | W/O |
| | | | solution of | | small drops | | emulsion |
| | | | doxorubicin | | and coarser | | Phase |
| | | | 50 mg in | | drops of | | separation |
| | | | 10 ml of | | 20-50 μm | | |
| | | | physiological | | | | |
| | | | saline | | | | |
| | | | supple- | | | | |
| | | | mented with | | | | |
| | | | dextan T40 | | | | |
| | | | at 2.5 g/50 ml | | | | |
| E16* | Cremophor ® | 0.5% | 2 ml of a | 1/4 | 2-5 | μm | W/O |
| | EL | | solution of | | | | emulsion |
| | | | doxorubicin | | | | Phase |
| | | | 50 mg in | | | | separation |
| | | | 10 ml of | | | | |
| | | | physiological | | | | |
| | | | saline | | | | |
| | | | supple- | | | | |
| | | | mented with | | | | |
| | | | dextan T40 | | | | |
| | | | at 3 g/50 ml | | | | |
| E17 | Tween ® 80 | 0.1% | Doxorubicin | 1/1 | 10 | μm | O/W |
| | | | (50 mg in | | | | emulsion |
| | | | 5 ml of | | | | Slight phase |
| | | | Xenetix ® | | | | separation at |
| | | | 250) | | | | 24 h |
| E18* | Tween ® 80 | 0.1% or | Doxorubicin | 1/1 | 10-100 | μm | O/W emulsion |
| | | 0.01% | (50 mg in | | | | |
| | | | 5 ml of | | | | |
| | | | physiological | | | | |
| | | | saline | | | | |
| | | | supple- | | | | |
| | | | mented with | | | | |
| | | | Tween ® 80 | | | | |
| | | | and 1% of | | | | |
| | | | PVP) | | | | |
| E19* | Tween ® 80 | 0.01% | Doxorubicin | 1/1 | 100-300 | μm | O/W |
| | | | HCl in 5 ml of | | | | emulsion |
| | | | glycerol at | | | | Immediate |
| | | | 2.5% | | | | phase |
| | | | | | | | separation |
| E19′ | Mixture of | | Doxorubicin | 1/3 | Heterogeneity | | Non- |
| | Tween ® 80 | 0.5% | (50 mg in | | | | emulsified |
| | and | | 2.5 ml of | | | | unstable |
| | Span ® 80 | 0.5% | Xenetix ® | | | | system |
| | | | 250) | | | | |
| E20 | CITHROL ® | 1% | Doxorubicin | 1/3 | Not | | W/O |
| | PG32IS | | 50 mg in | | measurable | | emulsion |
| | | | 2.5 ml of | | | | Instant and |
| | | | Xenetix ® | | | | violent phase |
| | | | 250) | | | | separation |
| E20′ | Pluronic ® | 5% | Doxorubicin | 1/4 | 10-100 | μm | O/W |
| | L101 | | 50 mg in | | | | emulsion |
| | | | 2.5 ml of | | | | Partial |
| | | | water | | | | phase |
| | | | | | | | separation |
| | | | | | | | before |
| | | | | | | | 18 hours |

*emulsion prepared with a densifying agent not in accordance with the invention: PVP (polyvinylpyrrolidone), Dextran T40, ioxaglic acid (Hexabrix ®) or glycerol

Emulsions Prepared without Surfactant and/or without Densifying Agent:

| | Nature of the | Aqueous | | | |
|---|---|---|---|---|---|
| Product | anti-cancer | phase/lipid | | | |
| number | agent used | phase ratio | Droplet sizes | | Observations |
| E21* | Doxorubicin | 1/1 | 10 | μm | O/W emulsion |
| | (50 mg in | | | | |
| | 5 ml of | | | | |
| | physiological | | | | |
| | saline) | | | | |
| E22 | Doxorubicin | 1/1 | 10 | μm | O/W emulsion |
| | (50 mg in | | | | |
| | 5 ml of | | | | |
| | Xenetix ® | | | | |
| | 250) | | | | |
| E23* | Doxorubicin | 1/3 or | Not | | W/O |
| | (50 mg in | 1/2 | measurable | | emulsion |
| | 2.5 ml of | | (greater than | | Immediate |
| | Xenetix ® | | 200 μm) | | phase |
| | 250) | | | | separation |
| E24* | Doxorubicin | 1/4 | 10 | μm | O/W |
| | (50 mg in | | | | emulsion |
| | 3 ml of | | | | Very viscous |
| | physiological | | | | and thick |
| | saline) | | | | emulsion: not |
| | | | | | usable in a |
| | | | | | clinical context |
| E25 | Epirubicin | 1/1 | 10 | μm | O/W emulsion |
| | (50 mg in 5 ml of | | | | |
| | lopamiron ® 250) | | | | |
| E26* | Epirubicin | 1/1 | 10-20 | μm | O/W emulsion |
| | (50 mg in 5 ml of | | | | |
| | physiological | | | | |
| | saline) | | | | |

*emulsion prepared without densifying agent

Span® 80 (Croda) is sorbitan monooleate. Tween® 80 (Croda), also called polysorbate 80, is PEG-20 sorbitan monooleate. Cremophor® EL (BASF) has, as chemical name: polyoxyl 35 Castor Oil. Cithrol® PG32IS is polyglyceryl-3-diisostearate. It is not therefore branched like the surfactants of formula (I).

Spans® other than Span® 80 (HLB=4.3) were tested: Span® 20 (HLB=8.6), Span® 65 (HLB=2.1), Span® 83 (HLB=3.7) and Span® 85 (HLB=1.8). The emulsions prepared with these surfactants all underwent phase separation immediately after they were prepared. The tests carried out with Pluronic® compounds (BASF) were also not conclusive since it was not possible to prepare an emulsion with these compounds.

Thus, all of the comparative emulsions obtained exhibited either insufficient stabilities, or a sense not in accordance with the invention.

### 3. In Vivo Evaluation of the Emulsions According to the Invention and Comparison with Emulsions not in Accordance with the Invention

3.1. Animal Model: Rat with Cancer Induced by Administration of N1-S1 Cells

### 3.1.1. Materials and Methods

The tumor induction method described in Garin et al. (Lab Anim. 2005 July; 39(3): 314-20) was used on female Sprague-Dawley rats (supplier Dépré or Janvier, France), anesthetized beforehand.

6×10⁶ N1-S1 rat hepatocellular carcinoma tumor cells (deposited at ATCC under the reference CRLL-1604™, also called Novikoff cells) suspended in 100 μl of IMDM medium (Iscove's Modified Dulbecco's Medium) were administered under the hepatic capsule of the left lateral lobe of the female rats, by slow injection over the course of approximately 50 seconds.

By way of information, the N1-S1 tumor cell line was initially obtained from a hepatoma induced by oral administration of 4-dimethylaminoazobenzene in a male Sprague-Dawley rat.

8 groups, each of 4 animals, were formed.

### Products Tested (One Product Per Group):

### Compositions According to the Invention:

| | Nature and | | |
|---|---|---|---|
| | amount of the | Nature and | |
| Products | anti-cancer | proportion of | Densifying |
| tested | agent used | surfactant used | agent used |
| E1 | 0.5 mg of | 1% (w/v) of PGPR | Yes (25 ul of |
| | doxorubicin* | | Xenetix ® 250) |
| E2 | 0.5 mg of | 1% (w/v) of PGPR | No |
| | doxorubicin* | | |
| E4 | 0.5 mg of | 1% (w/v) of PGPR | Yes (25 μl of |
| | epirubicin** | | Xenetix ® 250) |
| E11 | 0.5 mg of | 1% (w/v) of | Yes (25 μl of |
| | doxorubicin* | Cithrol ™ DPHS | Xenetix ® 250) |

*doxorubicin (Adriblastina ®, Pfizer)
**epirubicin (Farmorubicin ®, Pfizer)

The ratio between the aqueous phase and the lipid phase was ⅓ (25 μl of aqueous phase and 75 μl of Lipiodol®). The four emulsion compositions tested were inverse (W/O) emulsions.

Compositions not in Accordance with the Invention (without Surfactant):

| | Nature and | | | |
|---|---|---|---|---|
| | amount of the | | Aqueous | Sense |
| Products | anti-cancer | Densifying | phase/lipid | of the |
| tested | agent used | agent used | phase ratio | emulsion |
| E21 | 0.5 mg of | No | 1/1 | O/W |
| | doxorubicin* | | | |
| E22 | 0.5 mg of | Yes (50 μl of | 1/1 | O/W |
| | doxorubicin* | Xenetix ® 250) | | |
| E23 | 0.5 mg of | No | 1/3 | W/O |
| | doxorubicin* | | | |

*doxorubicin (Adriblastina ®, Pfizer)

### “Control” Products:

Doxorubicin alone (0.9% NaCl) or, where appropriate, epirubicin alone, was injected as a control in one of the groups of 4 rats.

### Administration of the Products:

These methods are well known to those skilled in the art who will therefore know themselves how to adjust certain parameters should that prove to be necessary.

The day before the TACE procedure (D-1), the animals were imaged by MRI (Bruker, 2.35 T) so as to verify the tumor growth. On the day of the TACE procedure (D0), the rats were again imaged in order to measure the size and then the volume (using image processing software) of the hepatic tumors before treatment.

7 days after having carried out the tumor induction method, the products (volume: 100 μl) were injected, via the gastroduodenal artery, into the pre-anesthetized animals.

Measurement of the Plasma Kinetics of the Anti-Cancer Agents Contained in these Emulsions after Injection Thereof:

Blood samples of 300 μl, at times 0, 5, 10, 20, 30 and 45 minutes after intra-arterial injection, were taken after catheterization of the carotid artery. 150 μl of plasma after centrifugation were then recovered and heparinized for the assaying of the anti-cancer agents. A study of the plasma kinetics of the anti-cancer agent was thus carried out.

The plasma doxorubicin assay was carried out in the heparinized rat plasma by high performance liquid chromatography (or HPLC), the chromatograph being equipped with a fluorescence detector. The plasma samples were prepared by precipitation in acidic medium (ammonium acetate, pH 3.5) with 40% of acetonitrile. 200 μl of rat plasma (lithium heparinate) were required. 10 μl of the extract were analyzed by reverse-phase HPLC on a Zorbax 300SB-C18 4.6×150 mm, 3.5 μm column (with a Zorbax 300SB-C18 4.6×12.5 mm, 5 μm precolumn) and fluorimetric detection (excitation wavelength 480 nm, emission wavelength 560 nm).

The analysis was carried out over the course of 30 minutes with a 5 mM ammonium acetate, pH 3.5/acetonitrile gradient. The samples were assayed via a calibration curve (DOX calibration range of 2 μg/l to 1600 μg/l-DOXoI calibration range of 2 μg/l to 400 μg/l). When the area of the HPLC peak was greater than the upper limit of quantification, 5 μl were injected instead of 10 μl in order to obtain an area of doxorubicin included in the range. The assays were carried out blind at times TO, 5 min, 10 min, 20 min, 30 min and 45 minutes.

### Histological Evaluations of the Samples of Tumor and of Healthy Liver:

The animals were euthanized by isoflurane gas anesthesia (5%) with 1 l/min of O₂. An autopsy was carried out and blood, plasma, tumor and healthy liver samples were taken in order to perform assays of doxorubicin and of epirubicin. The tumor and healthy liver samples taken were frozen (unfixed) for histological analysis of the tumor.

After processing (sections, fixing for H&E, etc.) of the slides on which samples of the specimens were placed, H&E (hematoxylin-eosin) staining was carried out in order to obtain a better differentiation between the tumor part and its healthy hepatic environment. Measurements of the fluorescence specific for the presence or absence of doxorubicin were carried out and the fluorescence levels were indicated on a scale of 1 (weak fluorescence) to 6 (strong fluorescence). This fluorescence is proportional to the amount of doxorubicin present in the tissue under consideration. All these techniques are well known to those skilled in the art.

### 3.1.2. Results Obtained

### 3.1.2.1. Plasma Kinetics of the Anti-Cancer Agents

It was possible to note the following points on the graph of FIG. 1 representing the averaged concentrations as a function of time:

The doxorubicin concentration peak is at 5 minutes for the eight groups of animals. The highest peak (i.e. the highest plasma concentration) is for the injection of doxorubicin alone (control Doxorubicin) with an average of 2447 μg/l. It is followed by the group which received the E22 product (emulsion sense O/W—1/1 ratio with Xenetix® 250 without surfactant) for which the average of the concentration peak is 1287 μg/l, i.e. a value practically two times lower than that obtained for the injection of doxorubicin alone, then by the group which received the E23 product (emulsion sense W/O—1/3 ratio with neither surfactant nor densifying agent), then by the group which received the E21 product (emulsion sense O/W-1/1 ratio with neither surfactant nor densifying agent). Between the sampling times of 10 and 45 min, the groups which received doxorubicin alone and the E21, E22 and E23 products follow the same decrease with lower values for the groups which received the E21, E22 and E23 products (at 45 min, “control doxorubicin alone” group=112 μg/l, “E21” group=106 μg/l, “E22” group=63 μg/l and “E23” group=143 μg/l).

For the groups which received the E1 (emulsion sense W/O—1/3 with Xenetix® 250 with PGPR as surfactant), E2 (emulsion sense W/O with PGPR, without densifying agent), E4 (emulsion sense W/O with epirubicin, PGPR and Xenetix® 250) and E11 (emulsion sense W/O with doxorubicin, Cithrol™ DPHS and Xenetix® 250) products, the doxorubicin concentration is much lower, this being the case on all the sampling points, than that of the groups which received doxorubicin alone or the E21, E22 and E23 products.

The analysis of the plasma kinetics shows that there is no plasma peak after injection of the compositions in accordance with the invention E1, E2, E11 (FIG. 1) and E4 (results not shown in FIG. 1).

| | | Percentage decrease in the |
|---|---|---|
| | | plasma concentration of the |
| | Plasma concentration | anti-cancer agent at |
| | of the anti-cancer | 5 minutes after injection of |
| | agent 5 minutes | the emulsion compared with |
| Products | after injection | the intra-arterial injection of |
| tested | (in μg/l) | the anti-cancer agent alone |
| E1 | 49 | 97.8% |
| E2 | 125 | 94.4% |
| E4 | 64 | 97.1% |
| E11 | 73 | 96.7% |
| Control | 2447 | |
| doxorubicin | | |
| alone* | | |

*Control epirubicin alone for the E4 emulsion

A reduction of more than 94% of the plasma doxorubicin level compared with the animals receiving only doxorubicin alone is thus observed for the female rats which received the stable W/O emulsions in accordance with the invention.

For the female rats which received the E21, E22 and E23 emulsions not in accordance with the invention, a plasma peak is observed 5 minutes after injection thereof.

| | | Percentage decrease in the |
|---|---|---|
| | | plasma concentration of the |
| | Plasma concentration | anti-cancer agent at |
| | of the anti-cancer | 5 minutes after injection of |
| | agent 5 minutes | the emulsion compared with |
| Products | after injection | the intra-arterial injection of |
| tested | (in μg/l) | the anti-cancer agent alone |
| E21 | 914 | 59.3% |
| E22 | 1287 | 42.7% |
| E23 | 1035 | 53.9% |
| Control | 2447 | |
| doxorubicin | | |
| alone | | |

A reduction of at best 59% of the plasma doxorubicin level compared with the animals receiving only doxorubicin alone is thus observed for the female rats which received the emulsions not in accordance with the invention.

The sense of the W/O emulsion and the stability thereof improved by using a surfactant of formula I (PGPR or PEG-30 dipolyhydroxystearate) make it possible to envision effective clinical use of a composition according to the invention.

### 3.1.2.2. Histological Evaluations of the Tumor and Healthy Liver Samples

The analysis of the slides and in particular of the H&E stainings makes it possible to demonstrate the presence of a tumor of the HCC type (polygonal cells joined in thick dense strings) in all the animals planted with N1S1 cells, clearly delimited with respect to the healthy tissue.

The fluorescence was measured using a Nikon Eclipse 80i ABS microscope equipped with a Nikon Intensilight C-HGFI precentered fiber illumination system. The images were viewed by means of the Hamamatsu NDP.view 2.5 visualizing software. The autofluorescence of the tissues was determined on sections of the control animals. This level of fluorescence is deduced from the levels of fluorescence observed for the tumor and healthy liver samples from the animals having received the emulsion compositions.

This measurement on these sections demonstrated differences in amounts of doxorubicin at the level of the tumor with, in the order of the classification performed (determined blind): E1 (score 6), E21 (score 1), E22 (score 3) and E23 (score 2).

The E1 emulsion therefore makes it possible to maintain the anti-cancer agent at the tumor level to a much larger extent than the emulsions not in accordance with the invention do.

The compositions of emulsions according to the invention demonstrate their great capacity for vectorizing anti-cancer agents since they allow these agents to remain in the tumor and not to depart into the vascular compartment.

3.2. Animal Model: Rabbit with Cancer Induced by Administration of VX2 Cancer Cells

### 3.2.1. Materials and Methods

The tumor induction method described by Hong et al. (Clin Cancer Res 2006; 12 (8): 2563-2567) was used on New Zealand rabbits (NZ; supplier Charles River, France), anesthetized beforehand.

Fragments of VX2 tumor tissues are implanted (a fragment of 25 mg/per liver of NZ rabbit) under the hepatic capsule of the left lateral lobe of the rabbits. These rabbits with a tumor in the left hepatic lobe will subsequently be referred to as “VX2 rabbits”.

3 groups, each of 6 animals, were formed.

### Products Tested (One Product Per Group):

E1 (Emulsion according to the invention)
E21 (Emulsion not in accordance with the invention)
“Control” product: Doxorubicin alone (0.9% NaCl) was injected as a control in one of the groups of 6 rabbits.

### Administration of the Products:

These methods are well known to those skilled in the art, who will therefore themselves know how to adjust certain parameters should that prove to be necessary.

On the day of the TACE procedure (D0), the animals were imaged by MRI (Bruker, 2.35 T) so as to verify the tumor growth and in order to measure the size and then the volume (using image processing software) of the hepatic tumors before treatment (D0).

19 days after having carried out the tumor induction method, the TACE is carried out. Thus, the products (volume: 300 μl) are injected into the pre-anesthetized animals, using a catheter guided by fluoroscopy (X-ray imaging) via the femoral artery and brought up to the artery feeding the hepatic tumor (injection only where selectivity has been achieved). The TACE is carried out in a dedicated room and under conditions close to clinical practice (interventional radiology).

Measurement of the Plasma Kinetics of the Anti-Cancer Agent Contained in these Emulsions after Injection Thereof:

Blood samples of 300 μl at times 0, 5, 15, 30 or 45 minutes after intra-arterial injection were taken after catheterization of the ear vein. 200 μl of plasma after centrifugation were then recovered and heparinized for assaying the anti-cancer agent. A study of the plasma kinetics of the anti-cancer agent was thus carried out.

The plasma doxorubicin assay was carried out in the heparinized plasma of VX2 rabbits under the same conditions as those described above. The analysis was also carried out under the same conditions as those described above.

### Histological Evaluations and Evaluations of the Amounts of Doxorubicin and of Lipiodol® Delivered on Tumor and Healthy Liver Samples:

The animals were euthanized by isoflurane (5%) gas anesthesia with 1 l/min of O₂ one day (D1) after carrying out the TACE (intra-arterial injection of the products under guidance by X-ray imaging). An autopsy was carried out and blood, plasma, tumor and healthy liver samples were taken in order to perform doxorubicin and Lipiodol® assays. The tumor and healthy liver samples taken were frozen (unfixed) for histological analysis of the tumor. For histological analysis of the tissues, the tumor and healthy liver samples were frozen (unfixed). Serial sections 7 μm thick were cut on a cryostat and then stored at −80° C.

H&E or HE topographical staining of the tissues consist of nuclear staining with hematoxylin then cytoplasmic staining with 1% eosin. The HE-stained tissues are directly analyzed on a white-light microscope.

The Lipiodol® is demonstrated by means of a silver impregnation method which is performed by incubating the tissues in a 2.5% silver nitrate solution, followed by 2 abundant rinses in distilled water. The tissues are then counter-stained with hematoxylin and observed under a white-light microscope.

To demonstrate the doxorubicin, the tissues are post-fixed in a 4% buffer formol solution, rinsed in PBS and then mounted using a mounting medium which preserves fluorescence (Prolong antifade reagent) and contains 4′,6′-diamidino-2-phenylindole (DAPI) which counter-stains the nuclei. The tissues are observed by epifluorescence by using a TRITC (tetramethylrhodamine) filter.

The amount of Lipiodol® or of doxorubicin and also the diffusion are evaluated semi-quantitatively on each stained slide using a scoring method.

The score scale is the following:
amount: score from 0 (absence) to 5 (diffuse presence),
diffusion: score of 0 (limited to the vascular lumen (for Lipiodol®), or absence of fluorescence (for doxorubicin)) to 3 (diffusion at a distance from the vessels of approximately 10 rows of cells and more).

Finally, the correlation of the distribution of doxorubicin and of Lipiodol® is evaluated according to the following grading:
Good: the compounds are detected at the level of the same tissue structures on serial sections (with the exception of a few rare structures, where only Lipiodol® is present).
Partial or very partial: only a few structures have the two compounds in common, the other structures contained Lipiodol® alone.
No: total absence of doxorubicin in the structures containing Lipiodol®.

The assaying of doxorubicin in the tumor was carried out by high performance liquid chromatography (or HPLC), the chromatograph being equipped with a fluorescence detector. The samples were prepared by grinding in an acetate buffer, pH 3.5, in a GentleMacs™ dissociator, then dilution (by a factor of ¼) in ground liver material (control), followed by precipitation in an acidic medium (ammonium acetate, pH 3.5) with 40% acetonitrile. 10 μl of the extract were analyzed by reverse-phase HPLC according to the conditions indicated above.

The analysis was carried out over the course of 34 minutes with a 5 mM ammonium acetate, pH 3.5/acetonitrile gradient. The samples were assayed via a calibration curve prepared in rabbit liver (from 100 ng/g to 20 000 ng/g).

The assaying of the Lipiodol® in the tumor is carried out by measuring the total iodine by X-ray fluorescence after grinding in water in a GentleMacs™ dissociator, against a calibration curve of rabbit liver which has been ground and doped with Lipiodol® (500 pgl/g to 24 000 μgl/g).

### 3.2.2. Results Obtained

### 3.2.2.1. Plasma Kinetics of the Anti-Cancer Agents

It was possible to note the following points on the graph of FIG. 2 representing the averaged concentrations as a function of time:

The plasma doxorubicin concentration peak is at 5 minutes for the four groups of animals. The highest peak is for the injection of doxorubicin alone (Control doxorubicin) with a mean (±SD) of 563±282 μg/l. It is followed by the group which received the E21 product (emulsion sense O/W—1/1 ratio without Xenetix® 250 without surfactant) for which the mean (±SD) of the concentration peak is 275±78 μg/l, then by the group which received the E1 product (emulsion sense O/W—1/3 ratio with 1% of surfactant and with densifying agent) with a mean (±SD) of 19±6 μg/l. Between the sampling times of 15 and 45 min, the groups which received doxorubicin alone and the E21 product follow the same decrease with lower values for the groups which received the E21 products. The results regarding the group which received the E1 product show a curve with an appearance that seems to be decreasing but very flattened (scale effect) owing to the very low concentrations found in the plasma, even at 5 minutes post-injection.

For the group which received the E1 product, the doxorubicin concentration is much lower, this being on all the sampling points, than that of the group which received the E21 product and is even more marked compared with doxorubicin alone.

The analysis of the plasma kinetics shows that there is no plasma peak after injection of the E1 composition in accordance with the invention (FIG. 2).

| | | Percentage decrease in the |
|---|---|---|
| | | plasma concentration of the |
| | Plasma concentration | anti-cancer agent at |
| | of the anti-cancer | 5 minutes after injection of |
| | agent 5 minutes | the emulsion compared with |
| Products | after injection | the intra-arterial injection of |
| tested | (in μg/l) | the anti-cancer agent alone |
| E1 | 19 ± 6 | 98% |
| E21 | 275 ± 78 | 82% |
| Control | 563 ± 282 | |
| doxorubicin | | |
| alone given | | |
| IA* | | |

*doxorubicin (Adriblastina ®, Pfizer)

A reduction of more than 98% of the plasma doxorubicin level compared with the animals receiving only doxorubicin alone is thus observed for the VX2 rabbits which received the W/O stable emulsions in accordance with the invention.

For the VX2 rabbits which received the E21 emulsion not in accordance with the invention, a plasma peak is observed 5 minutes after injection thereof.

A reduction of at best 51% of the plasma doxorubicin level compared with the animals receiving only doxorubicin alone is thus observed for the rabbits which received the emulsion not in accordance with the invention (FIG. 2).

These results confirm, on another animal model, that the sense of the W/O emulsion and the stability thereof improved by using a surfactant of formula I (PGPR) make it possible to envision effective clinical use of a composition according to the invention.

### 3.2.2. Delivery of the Anti-Cancer Agents

The determination of the amounts delivered after injection of the product in accordance with the invention E1 shows an amount of doxorubicin present at the tumor level that is significantly higher than the emulsion not in accordance with the invention E21 (Table 1).

TABLE 1
Intratumor concentration (μg/g)

| | E1 | E21 |
|---|---|---|
| Doxorubicin | 21 ± 9 | 8 ± 4 |

For the VX2 rabbits which received a W/O stable emulsion in accordance with the invention, a greater delivery of doxorubicin is thus observed than for the rabbits which received an emulsion not in accordance with the invention.

The amounts of Lipiodol® were also determined, and show a higher amount of Lipiodol® found in the tumor with the emulsion in accordance with the invention E1, compared with the emulsion not in accordance with the invention E21 (Table 2).

TABLE 2
Intratumor concentration (μg of iodine/g)

| | E1 | E21 |
|---|---|---|
| Lipiodol ® | 11257 ± 1912 | 6884 ± 2348 |

The determination of the doxorubicin and Lipiodol® concentrations made it possible to evaluate whether there was a correlation between these two measurements (FIG. 3). In FIG. 3, for the VX2 rabbits which received the W/O stable emulsion in accordance with the invention (E1), a high correlation is therefore observed between the doxorubicin and Lipiodol® concentrations in the tumor (r²=0.95). For the VX2 rabbits which received the E21 emulsion not in accordance with the invention, no correlation is observed.

For the VX2 rabbits which received the E1 emulsion, a higher concomitant delivery of doxorubicin and of Lipiodol® was therefore observed compared with the rabbits which received an emulsion not in accordance with the invention E21.

### 3.2.3. Histological Evaluations of the Tumor and Healthy Liver Samples

The analysis of the slides and in particular of the H&E stainings made it possible to demonstrate the presence of a tumor of the HCC type (polygonal cells joined in thick dense strings) in all the animals implanted with the VX2 tumor fragments, clearly delimited with respect to the healthy tissue.

On the cellular scale, doxorubicin is observed in the nuclei or in the nuclear debris, as its mechanism of action suggests. The doxorubicin distribution is evaluated semi-quantitatively (reading blind), the score demonstrates a difference in the amount of doxorubicin in the tumor after administration of the E1 product (score 3.5±0.84) and after administration of the E21 product (score 1.75±0.96). The E1 product delivers more doxorubicin into the tumor. With regard to Lipiodol®, the tumor distribution is evaluated semi-quantitatively after normalization with respect to the amount injected, the score does not show any difference in the amount of Lipiodol® in the tumor between the emulsions.

The E1 emulsion therefore makes it possible to deliver much more of the anti-cancer agent to the tumor than the emulsions not in accordance with the invention do. Thus, it is clearly demonstrated that the compositions of emulsions according to the invention have a large capacity for vectorizing anti-cancer agents since they allow these agents to remain in the tumor and not to depart into the vascular compartment.

Finally, the tissue correlation of the two compounds is good at the level of the tumor in the E1 group, but very partially for the E21 group, confirming the results obtained by assaying.

With regard to the toxicity of the products in the healthy liver, there is no difference in score for necrosis, inflammation, vascularity or biliary fibrosis/proliferation in the animals which received the E1 and E21 products and control animals (VX2 rabbits without injection).

1. A composition in the form of a water-in-oil emulsion comprising:
from 20% to 40% (v/v) of aqueous phase, in the form of droplets, comprising an anti-cancer agent,
from 60% to 80% (v/v) of lipid phase comprising an iodized oil and at least one surfactant of formula (I) in a proportion, by weight of surfactant relative to the total volume of the composition, of 0.3% to 5%, formula (I) of said surfactant being the following: 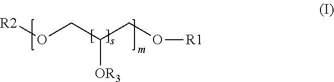
in which:
s is 0 or 1,
m is an integer from 2 to 30,
R₁ represents a group of formula (II) 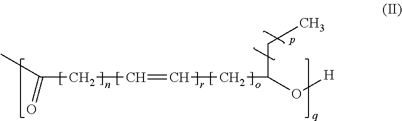
in which n is an integer from 4 to 10, o is an integer from 1 to 4, p is an integer from 3 to 7, q is an integer from 2 to 10, and r is 0 or 1,
R₂ represents a hydrogen atom or is identical to R₁, and
each R₃ independently represents a hydrogen atom or is identical to R₁.

2. The composition as claimed in claim 1, wherein each R₃ represents a hydrogen atom.

3. The composition as claimed in claim 1, said composition being stable.

4. The composition as claimed in claim 1, wherein the anti-cancer agent is selected from the group consisting of anthracyclines, platinum complexes, mitoxantrone, nemorubicin, mitomycin C, bleomycin, actinomycin D, irinotecan, 5-fluorouracil, sorafenib, sunitinib, regorafenib, brivanib, orantinib, linsitinib, erlotinib, cabozantinib, foretinib, tivantinib, fotemustine, tauromustine (TCNU), carmustine, cytosine C, cyclophosphonamide, cytosine arabinoside, paclitaxel, docetaxel, methotrexate, everolimus, PEG-arginine deiminase, the tegafur/gimeracil/oteracil combination, muparfostat, peretinoin, gemcitabine, bevacizumab and ramucirumab, floxuridine, GM-CSF, molgramostim, sargramostim, OK-432, interleukin-2, interleukin-4 and TNFalpha, 125I-labeled anti-CEA (carcinoembryonic antigen) antibodies, microspheres loaded with one of the foregoing compounds, radioelements and complexes of said radioelements with macrocyclic chelates, magnetic particles based on an iron compound a gadolinium chelate or both of the iron compound and the gadolinium chelate, radioactive microspheres, deoxyribonucleic acid sequences, ribonucleic acid sequences, and mixtures thereof.

5. The composition as claimed in claim 1, wherein the anthracyclines are selected from the group consisting of doxorubicin, epirubicin, nemorubicin and idarubicin.

6. The composition as claimed in claim 1, wherein the aqueous phase also comprises a densifying agent selected from the group consisting of nonionic iodinated contrast products and mixtures thereof.

7. The composition as claimed in claim 1, wherein the lipid phase also comprises a non-iodized oil selected from the group consisting of linseed oil, soybean oil, palm oil, coconut oil, caster oil, corn oil, cottonseed oil, peanut oil, sesame oil, sunflower oil, safflower oil, almond oil, olive oil, poppy oil, and an oil comprising a mixture of fatty acid triglycerides of the following formula: 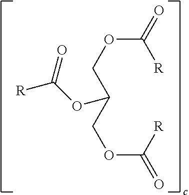
wherein R is an aliphatic chain comprising from 3 to 35 carbon atoms, and wherein more than 95% of said fatty acid triglycerides are C8 and/or C10.

8. The composition as claimed in claim 1, wherein the surfactant has an HLB of 1 to 8.

9. The composition as claimed in claim 1, wherein the surfactant is selected from the group consisting of polyglyceryl polyricinoleate and PEG-30 dipolyhydroxystearate.

10. The composition as claimed in claim 1, wherein the iodized oil comprises ethyl esters of iodized fatty acids of poppy oil or olive oil.

11. The composition as claimed in claim 1, wherein the size of the aqueous phase droplets is from 1 to 200 μm.

12. The composition as claimed in claim 1, having a viscosity at 20° C. that is from 100 to 200 mPa·s and/or a viscosity at 37° C. that is from 40 to 80 mPa·s

13. A method for treating cancer or metastases comprising administering to a patient the composition as claimed in claim 1.

14. A method for preparing the composition as claimed in claim 1, the method comprising the following steps:
a) mixing the at least one surfactant in the iodized oil to form the lipid phase, and
b) mixing the lipid phase with the aqueous solution comprising the anti-cancer agent,
thereby forming the composition.

15. Anti-cancer agent vector comprising a kit comprising an iodized oil and a surfactant of formula (I): 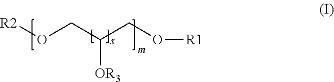
in which:
s is 0 or 1,
m is an integer from 2 to 30,
R₁ represents a group of formula (II) 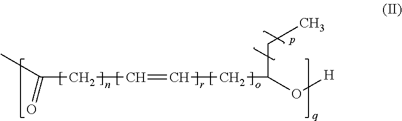
in which n is an integer from 4 to 10, o is an integer from 1 to 4, p is an integer from 3 to 7, q is an integer from 2 to 10, and r is 0 or 1,
R₂ represents a hydrogen atom or is identical to R₁, and
each R₃ independently represents a hydrogen atom or is identical to R₁.

16. Anti-cancer agent vector comprising the composition as claimed in claim 1.
